SEQUENCE LISTING (20 sequences)

SEQ ID NO: 1 - protein, 5 aa, Artificial Sequence
  Synthetic peptide
SYDMS

SEQ ID NO: 2 - protein, 17 aa, Artificial Sequence
  Synthetic peptide
LISGGGSQTYYAESVKG

SEQ ID NO: 3 - protein, 11 aa, Artificial Sequence
  Synthetic peptide
PSGHYFYAMDV

SEQ ID NO: 4 - protein, 11 aa, Artificial Sequence
  Synthetic peptide
RASQGISNWLA

SEQ ID NO: 5 - protein, 7 aa, Artificial Sequence
  Synthetic peptide
AASSLQS

SEQ ID NO: 6 - protein, 9 aa, Artificial Sequence
  Synthetic peptide
QQAESFPHT

SEQ ID NO: 7 - protein, 120 aa, Artificial Sequence
  Synthetic peptide
EVQLLESGGGLVQPGGSLRLSCAASGFTFSSYDMSWVRQAPGKGLEWVSLISGGGSQTYYAESVKGRFTISRDNSKNTLYLQMNSLRAEDTAVYFCASPSGHYFYAMDVWGQGTTVTVSS

SEQ ID NO: 8 - protein, 107 aa, Artificial Sequence
  Synthetic peptide
DIQMTQSPSSVSASVGDRVTITCRASQGISNWLAWYQQKPGKAPKLLIFAASSLQSGVPSRFSGSGSGTDFTLTISSLQPEDFATYYCQQAESFPHTFGGGTKVEIK

SEQ ID NO: 9 - protein, 472 aa, Artificial Sequence
  Synthetic peptide
MDMRVPAQLLGLLLLWLRGARCEVQLLESGGGLVQPGGSLRLSCAASGFTFSSYDMSWVRQAPGKGLEWVSLISGGGSQTYYAESVKGRFTISRDNSKNTLYLQMNSLRAEDTAVYFCASPSGHYFYAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 236 aa, Artificial Sequence
  Synthetic peptide
MDMRVPAQLLGLLLLWLRGARCDIQMTQSPSSVSASVGDRVTITCRASQGISNWLAWYQQKPGKAPKLLIFAASSLQSGVPSRFSGSGSGTDFTLTISSLQPEDFATYYCQQAESFPHTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 15 bp, Artificial Sequence
  Synthetic polynucleotide
agctatgacatgagc

SEQ ID NO: 12 - DNA, 51 bp, Artificial Sequence
  Synthetic polynucleotide
cttattagtggtggtggtagtcaaacatactacgcagaatccgtgaagggc

SEQ ID NO: 13 - DNA, 33 bp, Artificial Sequence
  Synthetic polynucleotide
cccagtggccactacttctacgctatggacgtc

SEQ ID NO: 14 - DNA, 33 bp, Artificial Sequence
  Synthetic polynucleotide
cgggcgagtcagggtattagcaactggttagcc

SEQ ID NO: 15 - DNA, 21 bp, Artificial Sequence
  Synthetic polynucleotide
gctgcatccagtttgcaaagt

SEQ ID NO: 16 - DNA, 27 bp, Artificial Sequence
  Synthetic polynucleotide
caacaggctgaaagtttccctcacact

SEQ ID NO: 17 - DNA, 360 bp, Artificial Sequence
  Synthetic polynucleotide
gaggtgcagctgttggagtctgggggaggcttggtacagcctggggggtccctgagactctcctgtgcagcctctggattcacctttagcagctatgacatgagctgggtccgccaggctccagggaaggggctggaatgggtctcacttattagtggtggtggtagtcaaacatactacgcagaatccgtgaagggccggttcaccatctccagagacaattccaagaacacgctgtatctgcaaatgaacagcctgagagccgaggacacggccgtatatttctgtgcgtcccccagtggccactacttctacgctatggacgtctggggccaagggaccacggtcaccgtctcctca

SEQ ID NO: 18 - DNA, 321 bp, Artificial Sequence
  Synthetic polynucleotide
gacatccagatgacccagtctccatcttccgtgtctgcatctgttggagacagagtcaccatcacttgtcgggcgagtcagggtattagcaactggttagcctggtatcagcagaaaccagggaaagcccctaagctcctgatctttgctgcatccagtttgcaaagtggggtcccatcaaggttcagcggcagtggatctgggacagatttcaccctcaccatcagcagcctgcagcctgaagattttgcaacttactattgtcaacaggctgaaagtttccctcacactttcggcggagggaccaaggtggagatcaaa

SEQ ID NO: 19 - DNA, 1416 bp, Artificial Sequence
  Synthetic polynucleotide
atggacatgagggtgcccgctcagctcctggggctcctgctgctgtggctgagaggtgcgcgctgtgaggtgcagctgttggagtctgggggaggcttggtacagcctggggggtccctgagactctcctgtgcagcctctggattcacctttagcagctatgacatgagctgggtccgccaggctccagggaaggggctggaatgggtctcacttattagtggtggtggtagtcaaacatactacgcagaatccgtgaagggccggttcaccatctccagagacaattccaagaacacgctgtatctgcaaatgaacagcctgagagccgaggacacggccgtatatttctgtgcgtcccccagtggccactacttctacgctatggacgtctggggccaagggaccacggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgtgcgaggagcagtacggcagcacgtaccgttgcgtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 20 - DNA, 708 bp, Artificial Sequence
  Synthetic polynucleotide
atggacatgagggtgcccgctcagctcctggggctcctgctgctgtggctgagaggtgcgcgctgtgacatccagatgacccagtctccatcttccgtgtctgcatctgttggagacagagtcaccatcacttgtcgggcgagtcagggtattagcaactggttagcctggtatcagcagaaaccagggaaagcccctaagctcctgatctttgctgcatccagtttgcaaagtggggtcccatcaaggttcagcggcagtggatctgggacagatttcaccctcaccatcagcagcctgcagcctgaagattttgcaacttactattgtcaacaggctgaaagtttccctcacactttcggcggagggaccaaggtggagatcaaacgaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

(19) United States
(12) Patent Application Publication
(10) Pub. No.: US 2022/0211784 A1
(43) Pub. Date: Jul. 7, 2022

(54) USE OF ONCOLYTIC VIRUSES IN THE NEOADJUVANT THERAPY OF CANCER

(71) Applicant: AMGEN INC., Thousand Oaks, CA (US)

(72) Inventor: Jennifer Lorraine GANSERT, Simi Valley, CA (US)

(21) Appl. No.: 17/599,028

(22) PCT Filed: Mar. 26, 2020
(86) PCT No.: PCT/US2020/024883

§ 371 Date: Sep. 28, 2021

Related U.S. Application Data
(60) Provisional application No. 62/825,929, filed on Mar. 29, 2019.
(60) Provisional application No. 62/882,013, filed on Aug. 2, 2019.
(60) Provisional application No. 62/898,889, filed on Sep. 11, 2019.

(51) Int. Cl. A61K 35/763; A61P 35/00; C07K 16/28; C12N 15/86

(52) U.S. Cl. CPC A61K 35/763; C12N 15/86; C07K 16/2818; A61P 35/00

(57) ABSTRACT

The invention relates to the use of an oncolytic virus in a neoadjuvant treatment regimen for the treatment of cancer.

### CROSS REFERENCE TO RELATED APPLICATIONS

This application is a national stage application under 35 U.S.C. § 371 of International Application No. PCT/US2020/024883, having an international filing date of Mar. 26, 2020; which claims the benefit under 35 U.S.C. 119(e) of U.S. Provisional Application No. 62/825,929 filed Mar. 29, 2019; U.S. Provisional Application No. 62/882,013 filed Aug. 2, 2019; and U.S. Provisional Application No. 62/898,889 filed Sep. 11, 2019, all of which are incorporated by reference herein in their entireties.

### REFERENCE TO THE SEQUENCE LISTING

This application contains a Sequence Listing in computer-readable form. The Sequence Listing is provided as a text file entitled A-2364-WO-PCT_SeqListing_ST25.txt, created Feb. 18, 2020, which is 15,346 bytes in size. The information in the electronic format of the Sequence Listing is incorporated herein by reference in its entirety.

### BACKGROUND

Although melanoma is amenable to early detection the prognosis of patients with high-risk primary melanoma or with macroscopic nodal involvement remains poor. The best option for patients with higher-risk melanoma (e.g., resectable melanoma) is to receive effective adjuvant therapy to reduce their chances of recurrence. Multiple systemic therapeutic agents have been tested as adjuvant therapy for melanoma with benefits seen. More recently ipilimumab at the high dose of 10 mg/kg has shown a significant improvement in terms of relapse free survival and overall survival for Stage 3 melanoma patients, but at a significant cost in terms of immune-related toxicities. Results from recent trials with immunotherapy (PD-1 inhibitors) and molecular targeted therapy (BRAF inhibitor+MEK inhibitor) have improved the management of adjuvant treatment for melanoma. As the results from these trials mature, new challenges in treatment decisions will arise—such as optimizing patients' selection through predictive and prognostic biomarkers, and management of treatment related adverse events, in particular immune related toxicities. Cancer Treat Rev. 2018 September; 69:101-111. doi: 10.1016/j.ctrv.2018.06.003. Epub 2018 Jun. 9.

It has been observed that achieving pCR following neoadjuvant chemotherapy is associated with significantly improved disease recurrence and survival rates in the context of triple negative and HER2+ breast cancers. Spring et al., Cancer Res Feb. 15, 2019 (79) (4 Supplement) GS2-03; DOI: 10.1158/1538-7445.SABCS18-GS2-03. Most recently, data presented by the International Neoadjuvant Melanoma Consortium (INMC) concluded that the ability to achieve pathologic complete response correlates with improved RFS. Menzies a et al, 2019 ASCO Annual Meeting). However, there remains a need for further research to evaluate the clinical utility of escalation/de-escalation strategies in the adjuvant setting based on neoadjuvant response for patients.

Thus, there remains a need for novel neoadjuvant regimens (such as those that utilize oncolytic viruses) that optimize the neoadjuvant, primary, and adjuvant treatments within those regimens.

### SUMMARY OF THE INVENTION

The present invention relates to a method for the treatment of cancer comprising administering a combination of an oncolytic virus and a first checkpoint inhibitor; surgically removing any remaining tumor; and administering a second checkpoint inhibitor, wherein the first and second checkpoint inhibitors may be the same or different.

The oncolytic virus used in the present invention may be an adenovirus, reovirus, measles, herpes simplex, Newcastle disease virus, senecavirus, or vaccinia virus. In particular embodiments, the oncolytic virus is an adenovirus, reovirus, herpes simplex, Newcastle disease virus, or vaccinia virus. In some embodiments, the oncolytic virus is a herpes simplex virus, such as a herpes simplex 1 virus (HSV-1). The HSV-1 may be modified such that it lacks functional ICP34.5 genes; lacks a functional ICP47 gene; and comprises a gene encoding a heterologous gene. In some embodiments, the heterologous gene is a cytokine, such as GM-CSF (e.g., human GM-CSF). In particular embodiments, the oncolytic virus is talimogene laherparepvec, RP1, RP2, or RP3. In another particular embodiment, the oncolytic virus is talimogene laherparepvec.

The first and second checkpoint inhibitor used in the present invention may be independently selected from the list comprising a CTLA-4 blocker, a PD-1 blocker, and a PD-L1 blocker. In some embodiments, the CTLA-4 blocker is an anti-CTLA-4 antibody, the PD-1 blocker is an anti-PD-1 antibody, and the PD-L1 blocker is an anti-PD-L1 antibody. The CTLA-4 blocker may be ipilimumab. The PD-1 blocker may be nivolumab, pembrolizumab, CT-011, AMP-224, cemiplimab, or an anti-PD-1 antibody comprising any one or more of SEQ ID NOs: 1-10. The PD-L1 blocker may be atezolizumab, avelumab, durvalumab, or BMS-936559.

Cancers that can be treated using the methods of the present invention include melanoma, breast cancer (e.g., triple negative breast cancer), renal cancer, bladder cancer, colorectal cancer, lung cancer, naso-pharyngeal cancer, pancreatic cancer, liver cancer, non-melanoma skin cancers, neuroendocrine tumors, T cell lymphoma (e.g., peripheral), or cancers of unknown primary origin, pediatric solid tumors with unresectable skin lesions. In some embodiments, the cancer is Stage 2, 3a, 3b, 3c, 3d or 41a melanoma.

The present invention also relates to kits comprising: [1] a herpes simplex virus lacking functional ICP34.5 genes, lacking a functional ICP47 gene, and comprising a gene encoding human GM-CSF; and [2] a package insert or label with directions to treat a cancer by administering a combination of an oncolytic virus and a first checkpoint inhibitor; surgically removing any remaining tumor; and administering a second checkpoint inhibitor, wherein said first and second checkpoint inhibitors may be the same or different. In some embodiments, the present invention relates to methods of manufacturing such kits.

### BRIEF DESCRIPTION OF THE DRAWINGS

FIG. 1 is the study schema of Amgen study 20120266 which is a A Phase 2, Multicenter, Randomized, Open-label Trial Assessing the Efficacy and Safety of Talimogene Laherparepvec Neoadjuvant Treatment Plus Surgery Versus Surgery Alone for Resectable, Stage IIIB to IVM1a Melanoma.

FIG. 2 is a Kaplan-Meier Plot depicting the time to regression-free survival (RFS) in the intent-to-treat (ITT) patient population at 1 year. All non-R0 resections at baseline were considered events (i.e., all recurrence+all non R0 resections). At 1 year, 33.5% of patients in Arm 1 and 21.9% in Arm 2 did not have evidence of disease recurrence (HR 0.73, P=0.048).

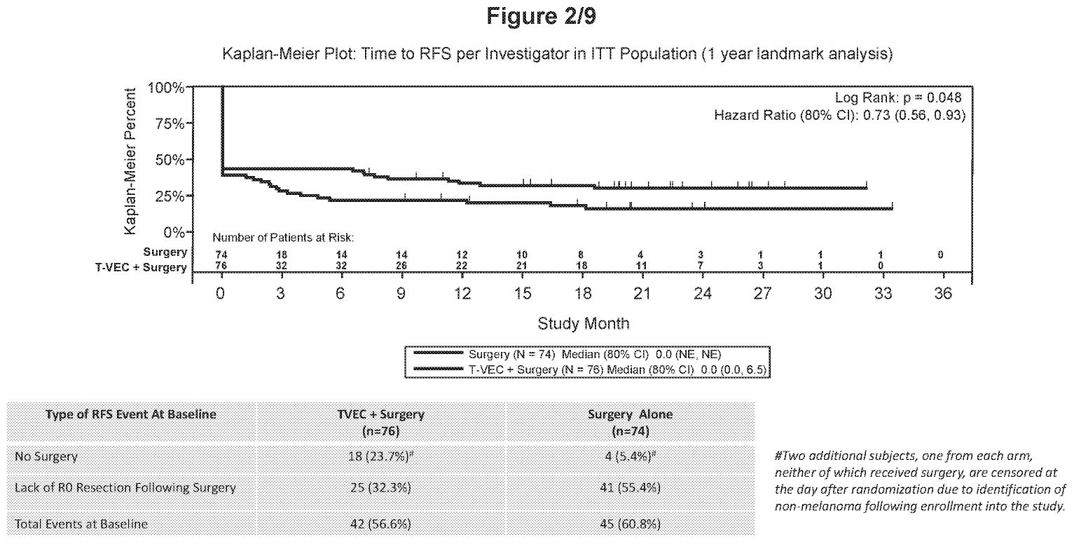

FIG. 3 is a Kaplan-Meier Plot depicting the time to regression-free survival (RFS) in the intent-to-treat (ITT) patient population at 2 years. All non-R0 resections at baseline were considered events (i.e., all recurrence+all non R0 resections). At 2 years, 29.5% of patients in Arm 1 and 16.5% in Arm 2 did not have evidence of disease recurrence (HR 0.75, P=0.070).

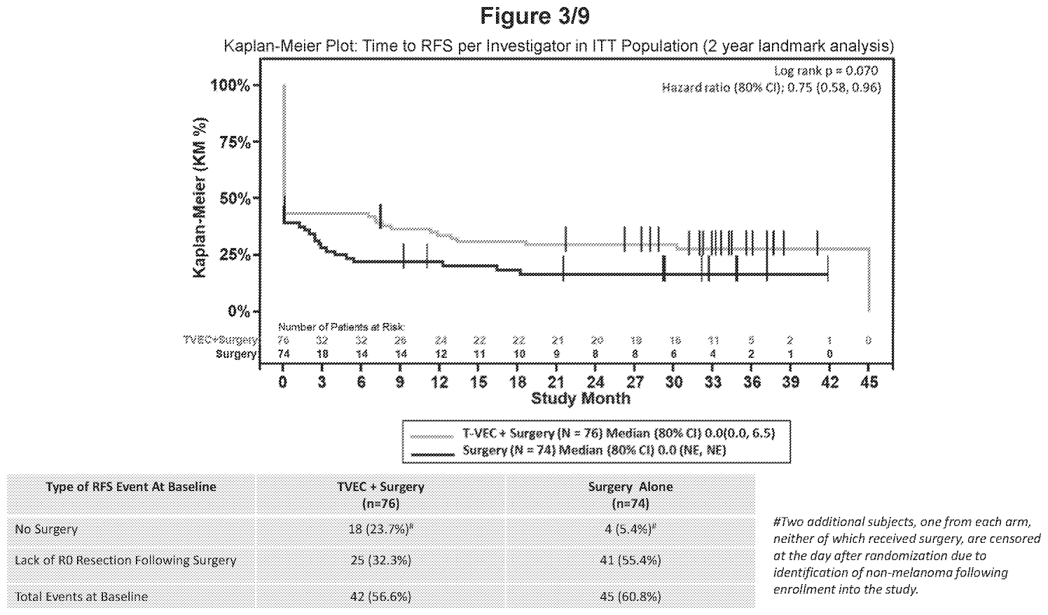

FIG. 4 is a Kaplan-Meier Plot depicting time to RFS in the ITT patient population, where non-R0 resections were not considered events at baseline (1 year landmark analysis). RFS was defined as the first of local, regional, or distant recurrence of melanoma or death due to any cause, following surgery. Subjects who did not receive surgery were considered events at baseline. At 1 year, 55.8% of pts in arm 1 and 39.3% in arm 2 remain recurrence free (HR 0.63, P=0.0024).

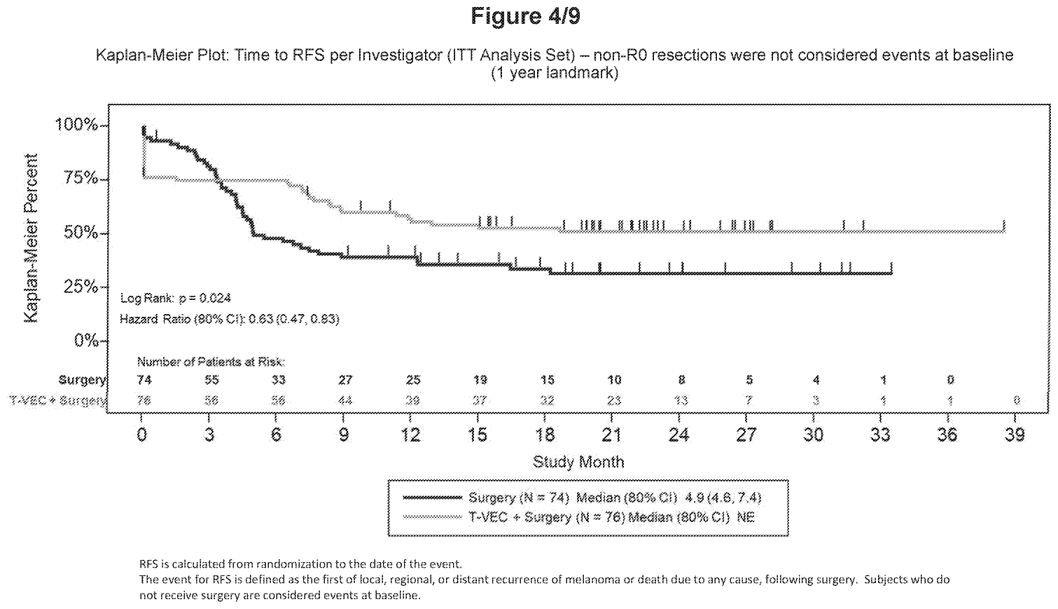

FIG. 5 is a Kaplan-Meier Plot depicting time to RFS in the ITT patient population, where non-R0 resections were not considered events at baseline (2 year landmark analysis). RFS was defined as the first of local, regional, or distant recurrence of melanoma or death due to any cause, following surgery. Subjects who did not receive surgery were considered events at baseline. At 2 years, 50.5% of patients in Arm 1 and 30.2% in Arm 2 did not have evidence of disease recurrence (HR 0.66, P=0.038).

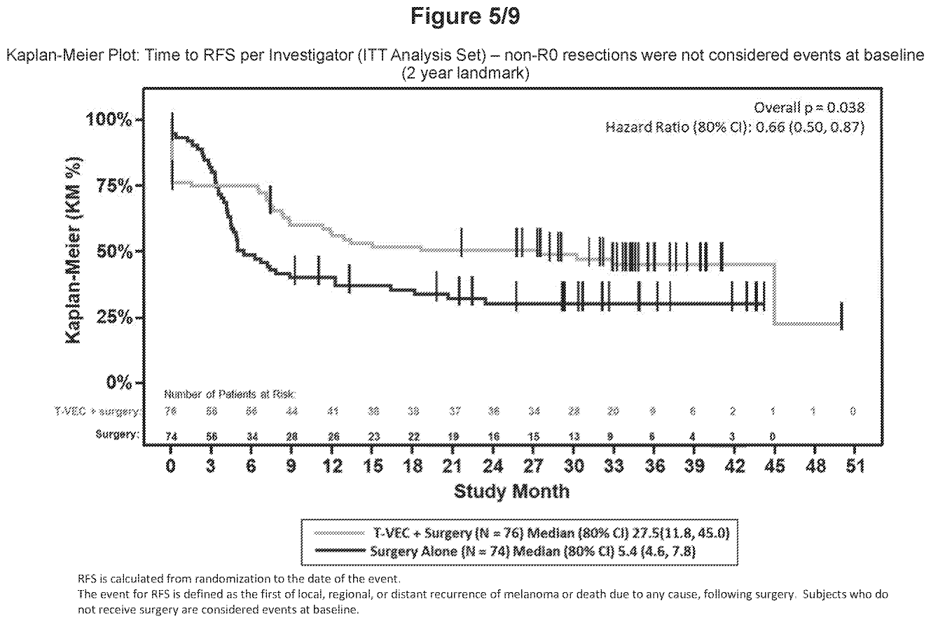

FIG. 6 is a Kaplan-Meier Plot depicting overall survival (OS) at 1 year. 95.9% of patients in arm 1 vs 85.8% patients in arm 2 were alive at the 1 year mark (HR 0.47, P=0.078).

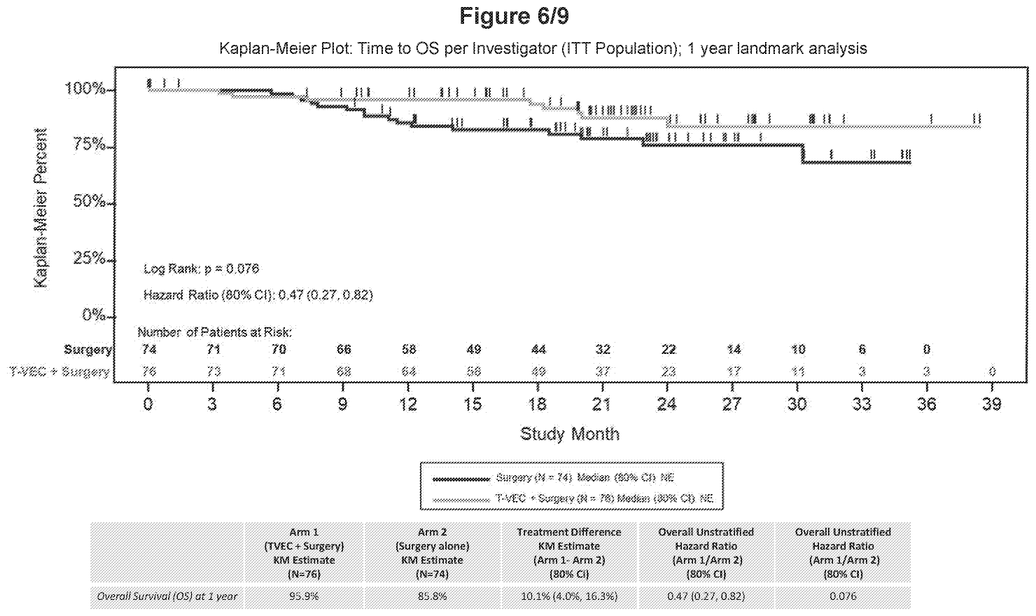

FIG. 7 is a Kaplan-Meier Plot depicting overall survival (OS) at 1 year. 88.9% of patients in Arm 1 and 77.4% of patients in Arm 2 were alive at the 2 year land mark (HR 0.49, P=0.050).

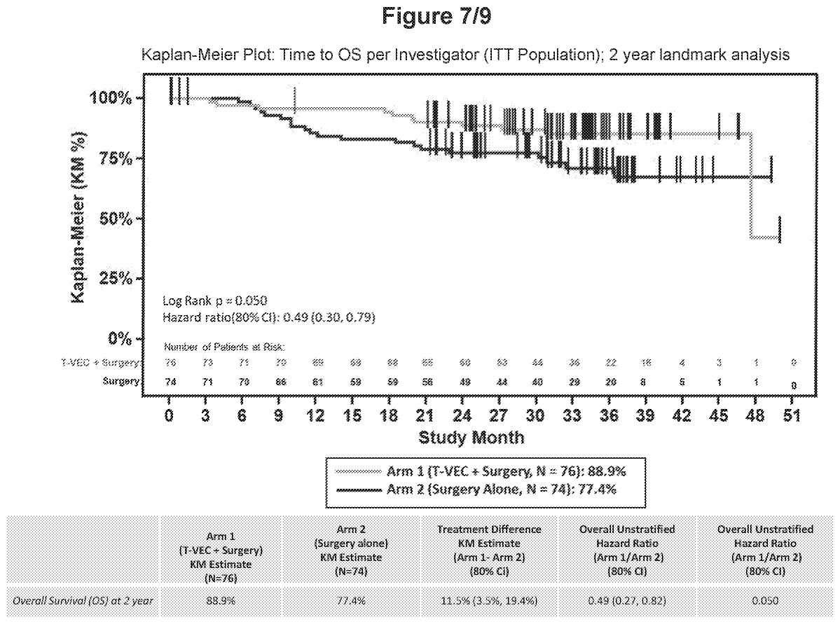

FIG. 8 illustrates that treatment with talimogene laherparepvec resulted in a 3-fold increase in intratumoral CD8+ cell density (P<0.001) and an increase in PD-L1 expression H-score of 17 units (P=0.038) in Arm 1. CD8+ density and PD-L1 H-score were also higher in Arm 1 after talimogene laherparepvec treatment compared to Arm 2 (both P<0.001)

FIG. 9 illustrates that, in Arm 1, the increase in intratumoral CD8+ cell density after talimogene laherparepvec treatment was correlated with longer RFS (sensitivity analysis) and longer OS.

### DETAILED DESCRIPTION OF THE INVENTION

As used herein, the term “immune checkpoint inhibitor” refers to molecules that totally or partially reduce, inhibit, interfere with or modulate one or more checkpoint proteins. Checkpoint proteins regulate T-cell activation or function. Numerous checkpoint proteins are known, such as CTLA-4 and its ligands CD80 and CD86; and PD-1 with its ligands PD-L1 and P-DL2 (Pardoll, Nature Reviews Cancer 12: 252-264, 2012). These proteins are responsible for co-stimulatory or inhibitory interactions of T-cell responses Immune checkpoint proteins regulate and maintain self-tolerance and the duration and amplitude of physiological immune responses. Immune checkpoint inhibitors include, e.g., antibodies or are derived from antibodies.

As used herein, the term “antibody” refers to a protein having a conventional immunoglobulin format, comprising heavy and light chains, and comprising variable and constant regions. For example, an antibody may be an IgG which is a “Y-shaped” structure of two identical pairs of polypeptide chains, each pair having one “light” (typically having a molecular weight of about 25 kDa) and one “heavy” chain (typically having a molecular weight of about 50-70 kDa). An antibody has a variable region and a constant region. In IgG formats, the variable region is generally about 100-110 or more amino acids, comprises three complementarity determining regions (CDRs), is primarily responsible for antigen recognition, and substantially varies among other antibodies that bind to different antigens. The constant region allows the antibody to recruit cells and molecules of the immune system. The variable region is made of the N-terminal regions of each light chain and heavy chain, while the constant region is made of the C-terminal portions of each of the heavy and light chains (Janeway et al., “Structure of the Antibody Molecule and the Immunoglobulin Genes”, Immunobiology: The Immune System in Health and Disease, 4^{th} ed. Elsevier Science Ltd./Garland Publishing, (1999)).

As used herein, the terms “patient” or “subject” are used interchangeably and mean a mammal, including, but not limited to, a human or non-human mammal, such as a bovine, equine, canine, ovine, or feline. Preferably, the patient is a human.

All clinical response evaluations discussed herein (e.g., ORR, DoR, etc. . . . ) are measured per the Response Evaluation Criteria in Solid Tumors (RECIST). See, Eisenhaurer E A, Therasse P, Bogaerts J, et al. New response evaluation criteria in solid tumours: Revised RECIST guideline (version 1.1). Eur J Cancer. 2009; 45: 228-247, which incorporated herein in its entirety.

As used herein, “objective response rate” is the incidence rate of either a confirmed complete response or partial response.

As used herein, “time to response” is the time from treatment to the date of the first confirmed objective response, per the modified RECIST.

As used herein, “duration of response” is the time from first confirmed objective response to confirmed disease progression per the modified RECIST or death, whichever occurs earlier.

As used herein, “progression free survival” is the time from treatment to the date of first of confirmed disease progression per modified RECIST criteria.

As used herein, “recurrence free survival” or “disease free survival” is the time from treatment (surgery) to the date of first recurrence or death.

As used herein, “event free survival” is the time from randomization until one of the following occurs: progression of disease that precludes surgery, local or distant recurrence, or death due to any cause

As used herein, “distant recurrence free survival” or “distant disease free survival” is the time from surgery to the first occurrence of the distant metastasis.

As used herein, “survival” refers to the patient remaining alive, and includes overall survival as well as progression free survival. 1-year survival rate and 2-year survival rate refers to the K-M estimate of the proportion of subjects alive at 12 month or 24 months.

As used herein, “extending survival” refers to increasing overall survival and/or progression free survival in a treated patient relative to a control treatment protocol, such as treatment with only ipilimumab. Survival is monitored for at least about one month, two months, four months, six months, nine months, or at least about 1 year, or at least about 2 years, or at least about 3 years, or at least about 4 years, or at least about 5 years, or at least about 10 years, etc., following the initiation of treatment or following the initial diagnosis.

As used herein, “reduce or inhibit” is the ability to cause an overall decrease of 20%, 30%, 40%, 50%, 60%, 70%, 75%, 80%, 85%, 90%, 95%, or greater. Reduce or inhibit can refer to the symptoms of the disorder being treated, the presence or size of metastases, or the size of the primary tumor.

Cancers can be divided into “Stages” based on the progression/advancement of the disease. Generally, the stages are divided into Stages 1, 2, 3, and 4, with some stage subdivisions wherein Stage 1 represents earlier stage disease and Stage 4 represent later/more advanced stage disease. For example, in the context of melanoma, patients with Stages 1 and 2 melanoma have localized disease, while those with stages III and IV melanoma have regional and distant metastatic disease, respectively. Although partially defined by the absence of regional disease, patients with Stage 2 melanoma with high-risk features (such as greater tumor thickness and presence of ulceration) may have a worse prognosis than patients with primary melanoma with more favorable features and limited occult regional metastatic (Stage 3A) disease. For example, patients with Stage 2C melanoma have worse expected five-year and 10-year survival than those with Stage 3A disease (82% and 75% vs 93% and 88%, respectively).

In addition, Stage 3 melanoma is divided into four subgroups based on tumor thickness, ulceration status and number of tumor-involved lymph nodes (and whether these were clinically occult versus clinically detected), as well as the presence or absence of non-nodal regional metastases. There are significant differences in prognosis across the four Stage 3 subgroups, with five-year melanoma specific survival (MSS) ranging from 93% for Stage 3A to 32% for Stage 3D disease. These rates are significantly better compared with five-year MSS for Stages 3A, 3B and 3C disease in the seventh edition (78%, 59%, and 40%, respectively), and will have a significant impact on clinical decision-making, patient counseling and clinical trial design.

Stage 4 Melanoma describes melanoma that has spread through the bloodstream to other parts of the body, such as distant locations on the skin or soft tissue, distant lymph nodes, or other organs like the lung, liver, brain, bone, or gastrointestinal tract. Stage 4 is further evaluated based on the location of distant metastasis. Stage 4a: The cancer has only spread to distant skin and/or soft tissue sites. Stage 4M1b: The cancer has spread to the lung. Stage 4M1c: The cancer has spread to any other location that does not involve the central nervous system. Stage 4M1d: The cancer has spread to the central nervous system, including the brain, spinal cord, and/or cerebrospinal fluid, or lining of the brain and/or spinal cord.

The terms “CD8 density,” “CD8+ density” or “CD8+ T-cell density” refer to the number of CD8+ T-cells present in a sample, e.g., in a tumor sample. In exemplary embodiments, a CD8+ T-cell density is the number of cells present in a sample, e.g., a 1 mm² sample (e.g., a punch biopsy) or a 1 mL (i.e., 1 cm³) sample (e.g., a liquid biopsy) of a tumor from a subject. In certain exemplary embodiments, a low CD8+ T-cell density (which is associated with a “cold” tumor) is less than about 3000 cells per 1 mm² or per 1 mL sample, less than about 2900 cells per 1 mm² or per 1 mL sample, less than about 2800 cells per 1 mm² or per 1 mL sample, less than about 2700 cells per 1 mm² or per 1 mL sample, less than about 2600 cells per 1 mm² or per 1 mL sample, less than about 2500 cells per 1 mm² or per 1 mL sample, less than about 2400 cells per 1 mm² or per 1 mL sample, less than about 2300 cells per 1 mm² or per 1 mL sample, less than about 2200 cells per 1 mm² or per 1 mL sample, less than about 2100 cells per 1 mm² or per 1 mL sample, less than about 2000 cells per 1 mm² sample, less than about 1900 cells per 1 mm² sample, less than about 1800 cells per 1 mm² or per 1 mL sample, less than about 1700 cells per 1 mm² or per 1 mL sample, less than about 1600 cells per 1 mm² or per 1 mL sample, less than about 1500 cells per 1 mm² or per 1 mL sample, less than about 1400 cells per 1 mm² or per 1 mL sample, less than about 1300 cells per 1 mm² or per 1 mL sample, less than about 1200 cells per 1 mm² or per 1 mL sample, less than about 1100 cells per 1 mm² or per 1 mL sample, less than about 1000 cells per 1 mm² or per 1 mL sample, less than about 900 cells per 1 mm² or per 1 mL sample, less than about 800 cells per 1 mm² or per 1 mL sample, less than about 700 cells per 1 mm² or per 1 mL sample, less than about 600 cells per 1 mm² or per 1 mL sample, less than about 500 cells per 1 mm² or per 1 mL sample, less than about 400 cells per 1 mm² or per 1 mL sample, less than about 300 cells per 1 mm² or per 1 mL sample, less than about 200 cells per 1 mm² or per 1 mL sample, or less than about 100 cells per 1 mm² or per 1 mL sample. In certain exemplary embodiments, a low CD8+ T-cell density is between about 3000 and 500 cells per 1 mm² or per 1 mL sample, between about 2900 and 500 cells per 1 mm² or per 1 mL sample, between about 2800 and 500 cells per 1 mm² or per 1 mL sample, between about 2700 and 500 cells per 1 mm² or per 1 mL sample, between about 2600 and 500 cells per 1 mm² or per 1 mL sample, between about 2500 and 500 cells per 1 mm² or per 1 mL sample, between about 2400 and 500 cells per 1 mm² or per 1 mL sample, between about 2300 and 500 cells per 1 mm² or per 1 mL sample, between about 2200 and 500 cells per 1 mm² or per 1 mL sample, between about 2100 and 500 cells per 1 mm² or per 1 mL sample, between about 2000 and 500 cells per 1 mm² or per 1 mL sample, between about 1900 and 500 cells per 1 mm² or per 1 mL sample, between about 1800 and 500 cells per 1 mm² or per 1 mL sample, between about 1700 and 500 cells per 1 mm² or per 1 mL sample, between about 1600 and 500 cells per 1 mm² or per 1 mL sample, 1500 and 500 cells per 1 mm² or per 1 mL sample, between about 1400 and 600 cells per 1 mm² or per 1 mL sample, between about 1300 and 700 cells per 1 mm² or per 1 mL sample, between about 1200 and 800 cells per 1 mm² or per 1 mL sample, between about 1100 and 900 cells per 1 mm² or per 1 mL sample, or between about 1050 and 950 cells per 1 mm² or per 1 mL sample. In certain exemplary embodiments, a low CD8+ T-cell density is between about 10 and 1000 cells per 1 mm² or per 1 mL sample, between about 20 and 900 cells per 1 mm² or per 1 mL sample, between about 30 and 800 cells per 1 mm² or per 1 mL sample, between about 40 and 700 cells per 1 mm² or per 1 mL sample, between about 50 and 600 cells per 1 mm² or per 1 mL sample, between about 60 and 500 cells per 1 mm² or per 1 mL sample, between about 70 and 400 cells per 1 mm² or per 1 mL sample, between about 80 and 300 cells per 1 mm² or per 1 mL sample, or between about 90 and 100 cells per 1 mm² or per 1 mL sample. In certain exemplary embodiments, a sample contains no detectable CD8+ T-cells.

### Use of Oncolytic Viruses in the Neoadjuvant Treatment of Cancer

The invention provides a method for the use of an oncolytic virus for the treatment of cancer. For example, the oncolytic virus may be used in a neoadjuvant treatment regimen for the treatment of cancer. In general, a neoadjuvant treatment is one that is given as a first step to shrink a tumor before a primary treatment is administered. Examples of primary treatment include, surgery, checkpoint inhibitor therapy (e.g., anti-PD-1, anti-PD-L1, and anti-CTLA-4), BRAF inhibitor therapy, MEK inhibitor therapy, chemotherapy, and combinations thereof. Examples of neoadjuvant therapy include chemotherapy, radiation therapy, hormone therapy, checkpoint inhibitor therapy, BRAF inhibitor therapy, MEK inhibitor therapy, and oncolytic virus therapy. In a particular embodiment, the primary treatment is surgery and the neoadjuvant treatment is an oncolytic virus.

In one embodiment, the present invention relates to the treatment of cancer wherein neoadjuvant oncolytic virus is administered, followed by primary treatment. In another embodiment, the present invention relates to the treatment of cancer wherein neoadjuvant oncolytic virus is administered, followed by primary treatment, followed by adjuvant therapy. In another embodiment, the present invention relates to the treatment of cancer wherein neoadjuvant oncolytic virus in combination with checkpoint inhibitor therapy is administered, followed by primary treatment, followed by adjuvant therapy. In one embodiment, the neoadjuvant therapy is an oncolytic virus such as an HSV-1 (e.g., talimogene laherparepvec, RP1, RP2, or RP3). In one embodiment, the neoadjuvant therapy is a combination of an oncolytic virus such as an HSV-1 (e.g., talimogene laherparepvec, RP1, RP2, or RP3) and a checkpoint inhibitor (e.g., anti-PD-1 such as pembrolizumab, nivolumab, or an anti-PD-1 antibody comprising any one or more of SEQ ID NOs: 1-10). In another embodiment, the neoadjuvant therapy is a combination of an oncolytic virus such as an HSV-1 (e.g., talimogene laherparepvec, RP1, RP2, or RP3) and a checkpoint inhibitor (e.g., anti-CTLA-4 such as ipilimumab). In another embodiment, the primary treatment is surgery. In yet another embodiment, the adjuvant therapy is checkpoint inhibitor therapy (e.g., anti-PD-1 such as pembrolizumab, nivolumab, or an anti-PD-1 antibody comprising any one or more of SEQ ID NOs: 1-10). In other embodiments, the oncolytic virus is talimogene laherparepvec.

Without being bound by a theory, the present invention utilizes combination therapy to increase the rate of pCR (pathological complete response), RFS, and/or OS without excessive toxicity. In addition, the neoadjuvant treatment regimens of the present invention can reduce or eliminate the amount and/or duration of primary treatment or adjuvant therapy, thus reducing the treatment cost and patient burden of treatment while maintaining clinical benefit.

### Patients Who are Anti-PD-1 Therapy Naïve

The present invention can be used to treat patients who are naïve to prior checkpoint inhibitor therapy (e.g., anti-PD-1 such as pembrolizumab or nivolumab)—i.e., the patient has not previously received prior checkpoint inhibitor therapy.

In a particular embodiment, the present invention relates to the treatment of cancer wherein a neoadjuvant oncolytic virus (e.g., talimogene laherparepvec) in combination with checkpoint inhibitor therapy (e.g., pembrolizumab or an anti-PD-1 antibody comprising any one or more of SEQ ID NOs: 1-10) is administered, followed by primary treatment (e.g., surgery), followed by checkpoint inhibitor (e.g., pembrolizumab or an anti-PD-1 antibody comprising any one or more of SEQ ID NOs: 1-10) adjuvant therapy. In some embodiments, the cancer is melanoma, breast cancer (e.g., triple negative breast cancer), renal cancer, bladder cancer, colorectal cancer, lung cancer, naso-pharyngeal cancer, pancreatic cancer, liver cancer, non-melanoma skin cancers, neuroendocrine tumors, T cell lymphoma (e.g., peripheral), or cancers of unknown primary origin, pediatric solid tumors with unresectable skin lesions. In some embodiments, the cancer is a Stage 3a, 3b, 3c, 3d, or 41a cancer. In a particular embodiment, the cancer is melanoma (e.g., a Stage 2 melanoma). In a particular embodiment, the cancer is melanoma (e.g., a Stage 3a, 3b, 3c, 3d, or 41a melanoma).

Suitable dosing can be determined by, e.g., a physician. In some embodiments, the neoadjuvant treatment comprises 1, 2, 3, 4, 5, 6, 7, 8, 9, or 10 doses. In a particular embodiment the neoadjuvant treatment comprises 1, 2, 3, 4, 5, 6, 7, 8, 9, or 10 doses of an oncolytic virus (e.g., talimogene laherparepvec, RP1, RP2, or RP3). In another embodiment the neoadjuvant treatment comprises 1, 2, 3, 4, 5, 6, 7, 8, 9, or 10 doses of a checkpoint inhibitor (e.g., pembrolizumab, nivolumab, or an anti-PD-1 antibody comprising any one or more of SEQ ID NOs: 1-10). In yet another embodiment the neoadjuvant treatment comprises a combination of 1, 2, 3, 4, 5, 6, 7, 8, 9, or 10 doses of an oncolytic virus (e.g., talimogene laherparepvec, RP1, RP2, or RP3) and 1, 2, 3, 4, 5, 6, 7, 8, 9, or 10 doses of a checkpoint inhibitor (e.g., pembrolizumab, nivolumab, or an anti-PD-1 antibody comprising any one or more of SEQ ID NOs: 1-10). In other embodiments, the neoadjuvant treatment comprises a combination of 1, 2, 3, 4, or 5 doses of an oncolytic virus (e.g., talimogene laherparepvec, RP1, RP2, or RP3) and 1, 2, or 3 doses of a checkpoint inhibitor (e.g., pembrolizumab, nivolumab, or an anti-PD-1 antibody comprising any one or more of SEQ ID NOs: 1-10). In yet other embodiments, the neoadjuvant treatment comprises a combination of 1, 2, or 3 doses of an oncolytic virus (e.g., talimogene laherparepvec, RP1, RP2, or RP3) and 1, 2, or 3 doses of a checkpoint inhibitor (e.g., pembrolizumab, nivolumab, or an anti-PD-1 antibody comprising any one or more of SEQ ID NOs: 1-10). In a particular embodiment, neoadjuvant treatment comprises a combination of talimogene laherparepvec and pembrolizumab. In a specific embodiment, neoadjuvant treatment comprises a combination of 3 doses of talimogene laherparepvec and 1 dose of pembrolizumab or nivolumab.

In some embodiments, the primary treatment comprises surgery.

In some embodiments, the adjuvant treatment comprises 1, 2, 3, 4, 5, 6, 7, 8, 9, 10, 11, 12, 13, 14, or 15 months of checkpoint inhibitor therapy (e.g., anti-PD-1 such as pembrolizumab, nivolumab, or an anti-PD-1 antibody comprising any one or more of SEQ ID NOs: 1-10). In other embodiments, the adjuvant treatment comprises 1, 2, 3, 4, 5, 6, 7, 8, 9, 10, 11, or 12 months of checkpoint inhibitor therapy (e.g., anti-PD-1 such as pembrolizumab, nivolumab, or an anti-PD-1 antibody comprising any one or more of SEQ ID NOs: 1-10). In some embodiments, the adjuvant treatment comprises 3, 6, 9, or 12 months of checkpoint inhibitor therapy (e.g., anti-PD-1 such as pembrolizumab, nivolumab, or an anti-PD-1 antibody comprising any one or more of SEQ ID NOs: 1-10). In a particular embodiment, the adjuvant treatment comprises treatment with 6 or 12 months of pembrolizumab, nivolumab, or an anti-PD-1 antibody comprising any one or more of SEQ ID NOs: 1-10.

### Patients Who Failed Previous Anti-PD-1 Therapy

In yet other embodiments of the present invention, the patient has failed (i.e., progressed after) prior checkpoint inhibitor (e.g., anti-PD-1 such as pembrolizumab or nivolumab) therapy—i.e., the patient's disease progressed after receiving checkpoint inhibitor therapy.

In a particular embodiment, the present invention relates to the treatment of cancer wherein neoadjuvant oncolytic virus (e.g., talimogene laherparepvec) in combination with checkpoint inhibitor therapy (e.g., anti-CTLA-4 such as ipilimumab) is administered, followed by primary treatment (e.g., surgery), followed by checkpoint inhibitor (e.g., anti-CTLA-4 such as ipilimumab) adjuvant therapy. In some embodiments, the cancer is melanoma, breast cancer (e.g., triple negative breast cancer), renal cancer, bladder cancer, colorectal cancer, lung cancer, naso-pharyngeal cancer, pancreatic cancer, liver cancer, non-melanoma skin cancers, neuroendocrine tumors, T cell lymphoma (e.g., peripheral), or cancers of unknown primary origin, pediatric solid tumors with unresectable skin lesions. In some embodiments, the cancer is a Stage 3a, 3b, 3c, 3d, or 41a cancer. In a particular embodiment, the cancer is melanoma (e.g., a Stage 3a, 3b, 3c, 3d, or 41a melanoma).

Suitable dosing can be determined by, e.g., a physician. In some embodiments, the neoadjuvant treatment comprises 1, 2, 3, 4, 5, 6, 7, 8, 9, or 10 doses. In a particular embodiment the neoadjuvant treatment comprises 1, 2, 3, 4, 5, 6, 7, 8, 9, or 10 doses of an oncolytic virus (e.g., talimogene laherparepvec, RP1, RP2, or RP3). In another embodiment the neoadjuvant treatment comprises 1, 2, 3, 4, 5, 6, 7, 8, 9, or 10 doses of a checkpoint inhibitor (e.g., anti-CTLA-4 such as ipilimumab). In yet another embodiment the neoadjuvant treatment comprises a combination of 1, 2, 3, 4, 5, 6, 7, 8, 9, or 10 doses of an oncolytic virus (e.g., talimogene laherparepvec, RP1, RP2, or RP3) and 1, 2, 3, 4, 5, 6, 7, 8, 9, or 10 doses of a checkpoint inhibitor (e.g., anti-CTLA-4 such as ipilimumab). In other embodiments, the neoadjuvant treatment comprises a combination of 1, 2, 3, 4, or 5 doses of an oncolytic virus (e.g., talimogene laherparepvec, RP1, RP2, or RP3) and 1, 2, 3, 4, or 5 doses of a checkpoint inhibitor (e.g., anti-CTLA-4 such as ipilimumab). In yet other embodiments, the neoadjuvant treatment comprises a combination of 1, 2, or 3 doses of an oncolytic virus (e.g., talimogene laherparepvec, RP1, RP2, or RP3) and 2, 3, or 4 doses of a checkpoint inhibitor (e.g., anti-CTLA-4 such as ipilimumab). In a particular embodiment, neoadjuvant treatment comprises a combination of talimogene laherparepvec and ipilimumab. In a specific embodiment, neoadjuvant treatment comprises a combination of 3 doses of talimogene laherparepvec and 4 doses of anti-CTLA-4 such as ipilimumab.

In some embodiments, the primary treatment comprises surgery.

In some embodiments, the adjuvant treatment comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 months of checkpoint inhibitor therapy (e.g., anti-CTLA-4 such as ipilimumab). In other embodiments, the adjuvant treatment comprises 1, 2, 3, 4, 5, 6, 7, 8, 9, 10, 11, 12, 13, 14, 15, 16, 17, 18, 19, 20, 21, 22, 23, or 24 months of checkpoint inhibitor therapy (e.g., anti-CTLA-4 such as ipilimumab). In some embodiments, the adjuvant treatment comprises 3, 6, 9, 12, 15, 18, 21, or 24 months of checkpoint inhibitor therapy (e.g., anti-CTLA-4 such as ipilimumab). In a particular embodiment, the adjuvant treatment comprises 12 or 24 months of ipilimumab treatment.

### Earlier Stage Melanoma Patients

In yet other embodiments of the present invention, the neoadjuvant treatment can be used to treat a patient with Stage 1 or Stage 2 cancer. In a specific embodiment, the patient has Stage 1 or Stage 2 melanoma. In another embodiment, the patient has Stage 1 melanoma. In another embodiment, the patient has Stage 2 melanoma.

In a particular embodiment, the present invention relates to the treatment of Stage 1 or Stage 2 cancer (e.g., melanoma) wherein neoadjuvant oncolytic virus (e.g., talimogene laherparepvec) is administered, followed by primary treatment (e.g., surgery), optionally followed by checkpoint inhibitor (e.g., anti-CTLA-4 such as ipilimumab, or anti-PD-1 such as pembrolizumab, nivolumab, or an anti-PD-1 antibody comprising any one or more of SEQ ID NOs: 1-10) adjuvant therapy. In some embodiments, the cancer is Stage 1 or Stage 2 melanoma, breast cancer (e.g., triple negative breast cancer), renal cancer, bladder cancer, colorectal cancer, lung cancer, naso-pharyngeal cancer, pancreatic cancer, liver cancer, non-melanoma skin cancers, neuroendocrine tumors, T cell lymphoma (e.g., peripheral), or cancers of unknown primary origin, pediatric solid tumors with unresectable skin lesions. In a particular embodiment, the cancer is Stage 2 melanoma.

Suitable dosing can be determined by, e.g., a physician. In some embodiments, the neoadjuvant treatment comprises 1, 2, 3, 4, 5, 6, 7, 8, 9, or 10 doses. In a particular embodiment the neoadjuvant treatment comprises 1, 2, 3, 4, 5, 6, 7, 8, 9, or 10 doses of an oncolytic virus (e.g., talimogene laherparepvec, RP1, RP2, or RP3). In other embodiments, the neoadjuvant treatment comprises 1, 2, 3, 4, 5, or 6 doses of an oncolytic virus (e.g., talimogene laherparepvec, RP1, RP2, or RP3). In yet other embodiments, the neoadjuvant treatment comprises 2, 3, 4, or 5 doses of an oncolytic virus (e.g., talimogene laherparepvec, RP1, RP2, or RP3). In a particular embodiment, neoadjuvant treatment comprises talimogene laherparepvec. In a specific embodiment, neoadjuvant treatment comprises 4 doses of talimogene laherparepvec.

In some embodiments, the primary treatment comprises surgery.

In some embodiments, the optional adjuvant treatment comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 months of checkpoint inhibitor therapy (e.g., anti-CTLA-4 such as ipilimumab). In other embodiments, the optional adjuvant treatment comprises 1, 2, 3, 4, 5, 6, 7, 8, 9, 10, 11, 12, 13, 14, 15, 16, 17, 18, 19, 20, 21, 22, 23, or 24 months of checkpoint inhibitor therapy (e.g., anti-CTLA-4 such as ipilimumab). In some embodiments, the optional adjuvant treatment comprises 3, 6, 9, 12, 15, 18, 21, or 24 months of checkpoint inhibitor therapy (e.g., anti-CTLA-4 such as ipilimumab). In a particular embodiment, the optional adjuvant treatment comprises 12 or 24 months of ipilimumab treatment.

In some embodiments, the optional adjuvant treatment comprises 1, 2, 3, 4, 5, 6, 7, 8, 9, 10, 11, 12, 13, 14, or 15 months of checkpoint inhibitor therapy (e.g., anti-PD-1 such as pembrolizumab, nivolumab, or an anti-PD-1 antibody comprising any one or more of SEQ ID NOs: 1-10). In other embodiments, the optional adjuvant treatment comprises 1, 2, 3, 4, 5, 6, 7, 8, 9, 10, 11, or 12 months of checkpoint inhibitor therapy (e.g., anti-PD-1 such as pembrolizumab, nivolumab, or an anti-PD-1 antibody comprising any one or more of SEQ ID NOs: 1-10). In some embodiments, the optional adjuvant treatment comprises 3, 6, 9, or 12 months of checkpoint inhibitor therapy (e.g., anti-PD-1 such as pembrolizumab, nivolumab, or an anti-PD-1 antibody comprising any one or more of SEQ ID NOs: 1-10). In a particular embodiment, the optional adjuvant treatment comprises treatment with 6 or 12 months of pembrolizumab, nivolumab, or anti-PD-1 antibody comprising any one or more of SEQ ID NOs: 1-10.

Patients with Low CD8+ Cell Density at Baseline

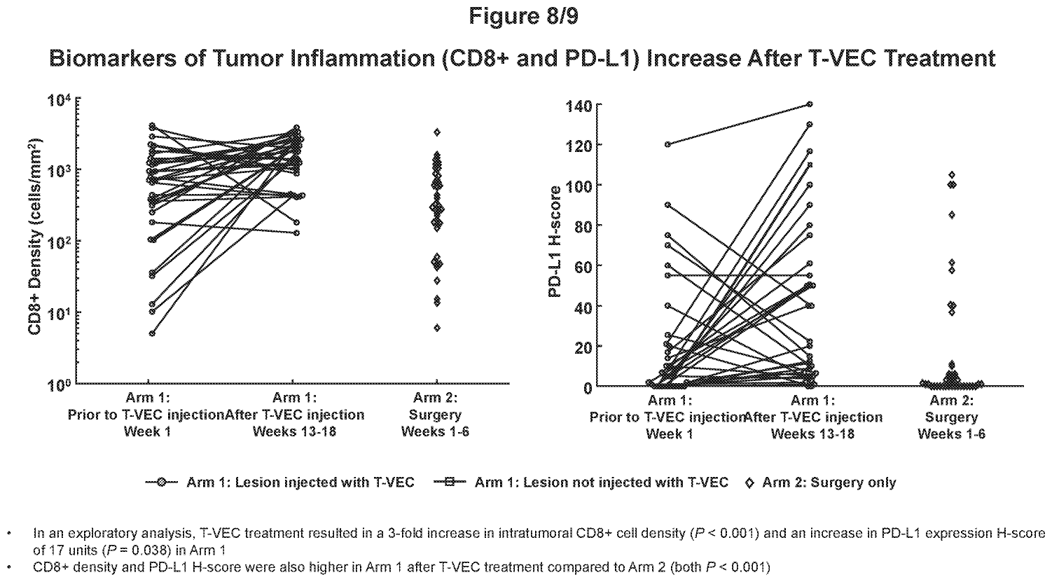

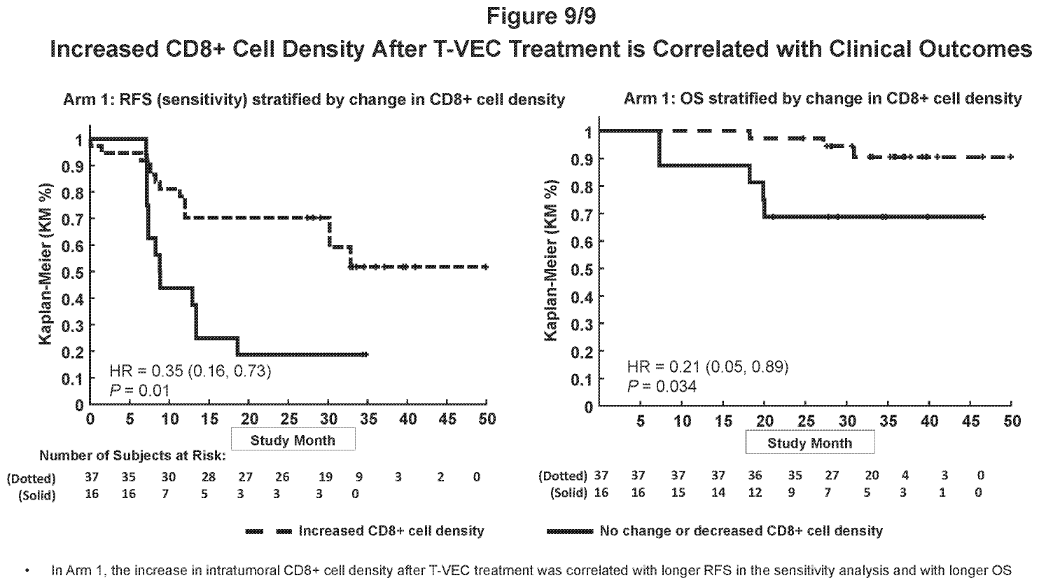

The present invention can be used to treat patients with low CD8+ cell density at baseline. It has been observed that treatment with talimogene laherparepvec results in an increase in intratumoral CD8+ cell density (see FIG. 8). Importantly, this increase in intratumoral CD8+ cell density after talimogene laherparepvec treatment correlates with longer RFS (sensitivity analysis) and longer OS (see FIG. 9). Thus, in some embodiments, the treatment regimens of the present invention are used to treat patients with “cold” tumors—i.e., tumors with low levels of intratumoral CD8+ cell density at baseline. Specifically, the administration of a neoadjuvant oncolytic virus (e.g., talimogene laherparepvec) to “cold” tumors improves the outcomes (e.g., RFS and OS) of subsequent primary treatment (e.g., surgery).

In certain embodiments, a patient with a “cold” tumor is selected for treatment with a treatment regimen of the present invention. In certain embodiments, the patient has a cold tumor with a CD8+ T-cell density less than or equal to about 3000, e.g., fewer than about 3000, about 2900, about 2800, about 2700, about 2600, about 2500, about 2400, about 2300, about 2200, about 2100, about 2000, about 1900, about 1800, about 1700, about 1600, about 1500, about 1400, about 1300, about 1200, about 1100, about 1000, about 900, about 800, about 700, about 600, or about 500 cells per 1 mm² or 1 mL (i.e., 1 cm³) sample. In some embodiments, the patient has a cold tumor with a CD8+ T-cell density less than or equal to about 1500, about 1400, about 1300, about 1200, about 1100, about 1000, about 900, about 800, about 700, about 600, or about 500 cells/mm².

### Oncolytic Viruses

In one embodiment, the oncolytic virus used in the present invention is an adenovirus, reovirus, measles, herpes simplex, Newcastle disease virus, senecavirus, or vaccinia virus. In a particular embodiment, the oncolytic virus is a herpes simplex virus (HSV). In exemplary aspects, the oncolytic virus is derived from a herpes simplex virus 1 (HSV-1) or herpes simplex 2 (HSV-2) strain, or from a derivative thereof, preferably HSV-1. Derivatives include inter-type recombinants containing DNA from HSV-1 and HSV-2 strains. Such inter-type recombinants are described in the art, for example in Thompson et al., (1998) Virus Genes 1(3); 275286, and Meignier et al., (1998) J. Infect. Dis. 159; 602614.

Herpes simplex virus strains may be derived from clinical isolates. Such strains are isolated from infected individuals, such as those with recurrent cold sores. Clinical isolates may be screened for a desired ability or characteristic such as enhanced replication in tumor and/or other cells in vitro and/or in vivo in comparison to standard laboratory strains, as described in U.S. Pat. Nos. 7,063,835 and 7,223,593, each of which are incorporated by reference in their entirety. In one embodiment the herpes simplex virus is a clinical isolate from a recurrent cold sore. Additional herpes simplex virus 1 virus strains include, but are not limited to, strain JS1, strain 17+, strain F, strain KOS, and strain Patton.

Examples of HSV genes that can be modified include virulence genes encoding proteins such as ICP34.5 (γ34.5). ICP34.5 acts as a virulence factor during HSV infection, limits replication in non-dividing cells and renders the virus non-pathogenic. Another HSV gene that can be modified is the gene encoding ICP47. ICP47 down-regulates major histocompatibility complex (MHC) class I expression on the surface of infected host cells and MHC Class I binding to transporter associated with antigen presentation (TAP). Such actions block antigenic peptide transport in the endoplasmic reticulum and loading of MHC class I molecules. Another HSV gene that can be modified is ICP6, the large subunit of ribonucleotide reductase, involved in nucleotide metabolism and viral DNA synthesis in non-dividing cells but not in dividing cells. Thymidine kinase, responsible for phosphorylating acyclovir to acyclovir-monophosphate, virion trans-activator protein vmw65, glycoprotein H, vhs, ICP43, and immediate early genes encoding ICP4, ICP27, ICP22 and/or ICP0, may be modified as well (in addition or alternative to the genes referenced above).

Herpes virus strains and how to make such strains are also described in U.S. Pat. Nos. 5,824,318; 6,764,675; 6,770,274; 7,063,835; 7,223,593; 7,749,745; 7,744,899; 8,273,568; 8,420,071; and 8,470,577; WIPO Publication Numbers WO199600007; WO199639841; WO199907394; WO200054795; WO2006002394; and WO201306795; Chinese Patent Numbers CN128303, CN10230334 and CN 10230335; Varghese and Rabkin, (2002) Cancer Gene Therapy 9:967-97, and Cassady and Ness Parker, (2010) The Open Virology Journal 4:103-108, which are incorporated by reference in their entirety.

In one embodiment, the oncolytic virus is talimogene laherparepvec (IMLYGIC®), derived from a clinical strain (HSV-1 strain JS1) deposited at the European collection of cell cultures (ECAAC) under accession number 01010209. In talimogene laherparepvec, the HSV-1 viral genes encoding ICP34.5 and ICP47 have been functionally deleted. Functional deletion of ICP47 leads to earlier expression of US11, a gene that promotes virus growth in tumor cells without decreasing tumor selectivity. The coding sequence for human GM-CSF, has been inserted into the viral genome at the former ICP34.5 sites (see Liu et al., Gene Ther 10: 292-303, 2003).

In some embodiments, the oncolytic virus is an HSV-1 which lacks a functional ICP34.5 encoding gene, lacks a functional ICP47 encoding gene, comprises a nucleic acid encoding Fms-related tyrosine kinase 3 ligand (FLT3L), and comprises a nucleic acid encoding interleukin-12 (IL-12). In some embodiments, the oncolytic virus is derived from a clinical strain (HSV-1 strain JS1) deposited at the European collection of cell cultures (ECAAC) under accession number 01010209.

Other examples of oncolytic viruses include RP1 (HSV-1/ICP34.5⁻/ICP47⁻/GM-CSF/GALV-GP R(−); RP2 (HSV-1/ICP34.5⁻/ICP47⁻/GM-CSF/GALV-GP R(−)/anti-CTLA-4 binder; and RP3 (HSV-1/ICP34.5⁻/ICP47⁻/GM-CSF/GALV-GP R(−)/anti-CTLA-4 binder/co-stimulatory ligands (e.g., CD40L, 4-1BBL, GITRL, OX40L, ICOSL)). In such oncolytic viruses, GALV (gibbon ape leukemia virus) has been modified with a specific deletion of the R-peptide, resulting in GALV-GP R(−). Such oncolytic viruses are discussed in WO2017118864, WO2017118865, WO2017118866, WO2017118867, and WO2018127713A1, each of which is incorporated by reference in its entirety.

Additional examples of oncolytic viruses include NSC-733972, HF-10, BV-2711, JX-594, Myb34.5, AE-618, Brainwel™, and Heapwel™, Cavatak® (coxsackievirus, CVA21), HF-10, Seprehvir®, Reolysin®, enadenotucirev, ONCR-177, and those described in U.S. Pat. No. 10,105,404, WO2018006005, WO2018026872A1, and WO2017181420, each of which is incorporated by reference in its entirety.

Further examples of oncolytic viruses include:

[A] G207, an oncolytic HSV-1 derived from wild-type HSV-1 strain F having deletions in both copies of the major determinant of HSV neurovirulence, the ICP 34.5 gene, and an inactivating insertion of the E. coli lacZ gene in UL39, which encodes the infected-cell protein 6 (ICP6), see Mineta et al. (1995) Nat Med. 1:938-943.

[B] OrienX010, a herpes simplex virus with deletion of both copies of γ34.5 and the ICP47 genes as well as an interruption of the ICP6 gene and insertion of the human GM-CSF gene, see Liu et al., (2013) World Journal of Gastroenterology 19(31):5138-5143.

[C] NV1020, a herpes simples virus with the joint region of the long (L) and short (S) regions is deleted, including one copy of ICP34.5, UL24, and UL56.34,35. The deleted region was replaced with a fragment of HSV-2 US DNA (US2, US3 (PK), gJ, and gG), see Todo, et al. (2001) Proc Natl Acad Sci USA. 98:6396-6401.

[D] M032, a herpes simplex virus with deletion of both copies of the ICP34.5 genes and insertion of interleukin 12, see Cassady and Ness Parker, (2010) The Open Virology Journal 4:103-108.

[E] ImmunoVEX HSV2, is a herpes simplex virus (HSV-2) having functional deletions of the genes encoding vhs, ICP47, ICP34.5, UL43 and US5.

[F] OncoVEX^{GALV/CD}, is also derived from HSV-1 strain JS1 with the genes encoding ICP34.5 and ICP47 having been functionally deleted and the gene encoding cytosine deaminase and gibbon ape leukaemia fusogenic glycoprotein inserted into the viral genome in place of the ICP34.5 genes.

The herpes simplex viruses of the invention may also comprise one or more heterologous genes. Heterologous gene refers to a gene to be introduced to the genome of a virus, wherein that gene is not normally found in the virus' genome or is a homolog of a gene expressed in the virus from a different species which has a different nucleic acid sequence and acts via a different biochemical mechanism. The heterologous genes may encode one or more proteins, for example, a cytotoxin, an immunomodulatory protein (i.e., a protein that either enhances or suppresses a host immune response to an antigen), a tumor antigen, prodrug activator, a tumor suppressor, a prodrug converting enzyme, proteins capable of causing cell to cell fusion, a TAP inhibitor antisense RNA molecule, or a ribozyme. Examples of immunomodulatory proteins include, for example, cytokines. Cytokines include an interleukins, such as IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-20; α, β or γ-interferons, tumor necrosis factor alpha (TNFα), CD40L, granulocyte macrophage colony stimulating factor (GM-CSF), macrophage colony stimulating factor (M-CSF), and granulocyte colony stimulating factor (G-CSF), chemokines (such as neutrophil activating protein (NAP), macrophage chemoattractant and activating factor (MCAF), RANTES, and macrophage inflammatory peptides MIP-1a and MIP-1b), complement components and their receptors, immune system accessory molecules (e.g., B7.1 and B7.2), adhesion molecules (e.g., ICAM-1, 2, and 3), and adhesion receptor molecules. Tumor antigens include the E6 and E7 antigens of human papillomavirus, EBV-derived proteins, mucins, such as MUC1, melanoma tyrosinase, and MZ2-E. Pro-drug activators include nitroeductase and cytochrome p450, tumour suppressors include p53. a prodrug converting enzymes include cytosine deaminase. Proteins capable of causing cell to cell fusion include gibbon ape leukaemia fusogenic glycoprotein. TAP inhibitors include the bovine herpesvirus (BHV) UL49.5 polypeptide. Antisense RNA molecules that can be used to block expression of a cellular or pathogen mRNA. RNA molecules that can be a ribozyme (e.g., a hammerhead or a hairpin-based ribozyme) designed either to repair a defective cellular RNA, or to destroy an undesired cellular or pathogen-encoded RNA.

Also included is insertion of multiple viral genes into the herpes simplex genome, such as insertion of one or more copies of the gene encoding viral protein Us11.

Talimogene laherparepvec, HSV-1 [strain JS1] ICP34.5-/ICP47-/hGM-CSF, (previously known as OncoVEX^{GM-CSF}), is an intratumorally delivered oncolytic immunotherapy comprising an immune-enhanced HSV-1 that selectively replicates in solid tumors. (Lui et al., Gene Therapy, 10:292-303, 2003; U.S. Pat. Nos. 7,223,593 and 7,537,924). The HSV-1 was derived from Strain JS1 as deposited at the European collection of cell cultures (ECAAC) under accession number 01010209. In talimogene laherparepvec, the HSV-1 viral genes encoding ICP34.5 have been functionally deleted. Functional deletion of ICP34.5, which acts as a virulence factor during HSV infection, limits replication in non-dividing cells and renders the virus non-pathogenic. The safety of ICP34.5-functionally deleted HSV has been shown in multiple clinical studies (MacKie et al, Lancet 357: 525-526, 2001; Markert et al, Gene Ther 7: 867-874, 2000; Rampling et al, Gene Ther 7:859-866, 2000; Sundaresan et al, J. Virol 74: 3822-3841, 2000; Hunter et al, J Virol August; 73(8): 6319-6326, 1999). In addition, ICP47 (which blocks viral antigen presentation to major histocompatibility complex class I and II molecules) has been functionally deleted from talimogene laherparepvec. Functional deletion of ICP47 also leads to earlier expression of US11, a gene that promotes virus growth in tumor cells without decreasing tumor selectivity. The coding sequence for human GM-CSF, a cytokine involved in the stimulation of immune responses, has been inserted into the viral genome of talimogene laherparepvec. The insertion of the gene encoding human GM-CSF is such that it replaces nearly all of the ICP34.5 gene, ensuring that any potential recombination event between talimogene laherparepvec and wild-type virus could only result in a disabled, non-pathogenic virus and could not result in the generation of wild-type virus carrying the gene for human GM-CSF. The HSV thymidine kinase (TK) gene remains intact in talimogene laherparepvec, which renders the virus sensitive to anti-viral agents such as acyclovir. Therefore, acyclovir can be used to block talimogene laherparepvec replication, if necessary.

Talimogene laherparepvec produces a direct oncolytic effect by replication of the virus in the tumor, and induction of an anti-tumor immune response enhanced by the local expression of GM-CSF. Since melanoma is a disseminated disease, this dual activity is beneficial as a therapeutic treatment. The intended clinical effects include the destruction of injected tumors, the destruction of local, locoregional, and distant uninjected tumors, a reduction in the development of new metastases, a reduction in the rate of overall progression and of the relapse rate following the treatment of initially present disease, and prolonged overall survival.

Talimogene laherparepvec has been tested for efficacy in a variety of in vitro (cell line) and in vivo murine tumor models and has been shown to eradicate tumors or substantially inhibit their growth at doses comparable to those used in clinical studies. Nonclinical evaluation has also confirmed that GM-CSF enhances the immune response generated, enhancing both injected and uninjected tumor responses, and that increased surface levels of MHC class I molecules result from the deletion of ICP47. Talimogene laherparepvec has been injected into normal and tumor-bearing mice to assess its safety. In general, the virus has been well tolerated, and doses up to 1×10⁸ PFU/dose have given no indication of any safety concerns. (See, for example, Liu et al., Gene Ther 10: 292-303, 2003)

Clinical studies have been or are being conducted in several advanced tumor types (advanced solid tumors, melanoma, squamous cell cancer of the head and neck, and pancreatic cancer), with over 400 subjects treated with talimogene laherparepvec (see, for example, Hu et al., Clin Can Res 12: 6737-6747, 2006; Harrington et al., J Clin Oncol. 27(15a):abstract 6018, 2009; Kaufman et al., Ann Surgic Oncol. 17: 718-730, 2010; Kaufman and Bines, Future Oncol. 6(6): 941-949, 2010). Clinical data indicate that talimogene laherparepvec has the potential to provide overall clinical benefit to patients with advanced melanoma. In particular, a high rate of complete response was achieved in Stage 3c to Stage 4 melanoma (Scenzer et al., J. Clin. Oncol. 271(12):907-913, 2009). In addition, responses were observed in both injected and uninjected sites, including visceral sites.

Talimogene laherparepvec is administered by intratumoral injection into injectable cutaneous, subcutaneous, and nodal tumors at a dose of up to 4.0 ml of 10⁶ plaque forming unit/mL (PFU/mL) at day 1 of week 1 followed by a dose of up to 4.0 ml of 10⁸ PFU/mL at day 1 of week 4, and every 2 weeks (±3 days) thereafter. The recommended volume of talimogene laherparepvec to be injected into the tumor(s) is dependent on the size of the tumor(s) and should be determined according to the injection volume guideline in Table 1.

TABLE 1
Talimogene Laherparepvec Injection Volume
Guidelines Based on Tumor Size

| Tumor Size (longest dimension) | Maximum Injection Volume |
|---|---|
| ≥5.0 cm | 4.0 ml |
| >2.5 cm to 5.0 cm | 2.0 ml |
| >1.5 cm to 2.5 cm | 1.0 ml |
| >0.5 cm to 1.5 cm | 0.5 ml |
| ≤0.5 cm | 0.1 ml |

All reasonably injectable lesions (cutaneous, subcutaneous and nodal disease that can be injected with or without ultrasound guidance) should be injected with the maximum dosing volume available on an individual dosing occasion. On each treatment day, prioritization of injections is recommended as follows: any new injectable tumor that has appeared since the last injection; by tumor size, beginning with the largest tumor; any previously uninjectable tumor(s) that is now injectable.

The duration of therapy will continue for as long as medically indicated or until a desired therapeutic effect (e.g., those described herein) is achieved. For example, patients can be treated with talimogene laherparepvec until complete response, all injectable tumors have disappeared, disease progression per the Response Evaluation Criteria in Solid Tumors (RECIST). Due to the mechanism of action, patients may experience growth in existing tumors or the appearance of new tumors prior to maximal clinical benefit of talimogene laherparepvec. Therefore, it is anticipated that dosing should be continued for at least 6 months from the time of initial dose provided that the subject has no evidence of clinically significant deterioration of health status requiring discontinuation of treatment and is able to tolerate the treatment. However, the course of treatment for any individual patient can be modified in clinical practice.

### Primary Treatments

The primary treatment of any of the treatment regimens of the present invention described herein may be surgery, checkpoint inhibitor therapy (e.g., anti-PD-1, anti-PD-L1, and anti-CTLA-4), BRAF inhibitor therapy, MEK inhibitor therapy, and combinations thereof. In a particular embodiment, the primary treatment is surgery.

### Adjuvant Therapies

The adjuvant therapy of any of the treatment regimens of the present invention described herein may be a checkpoint inhibitor therapy (e.g., anti-PD-1, anti-PD-L1, and anti-CTLA-4), BRAF inhibitor therapy, MEK inhibitor therapy, and combinations thereof. In a particular embodiment, the adjuvant therapy is a checkpoint inhibitor (e.g., anti-CTLA4 such as ipilimumab; or anti-PD-1 such as pembrolizumab, nivolumab, or an anti-PD-1 antibody comprising any one or more of SEQ ID NOs: 1-10).

The immune system has multiple inhibitory pathways that are critical for maintaining self-tolerance and modulating immune responses. In T-cells, the amplitude and quality of response is initiated through antigen recognition by the T-cell receptor and is regulated by immune checkpoint proteins that balance co-stimulatory and inhibitory signals.

Cytotoxic T-lymphocyte associated antigen 4 (CTLA-4) is an immune checkpoint protein that down-regulates pathways of T-cell activation (Fong et al., Cancer Res. 69(2):609-615, 2009; Weber Cancer Immunol. Immunother, 58:823-830, 2009). Blockade of CTLA-4 has been shown to augment T-cell activation and proliferation. Inhibitors of CTLA-4 include anti-CTLA-4 antibodies. Anti-CTLA-4 antibodies bind to CTLA-4 and block the interaction of CTLA-4 with its ligands CD80/CD86 expressed on antigen presenting cells and thereby blocking the negative down regulation of the immune responses elicited by the interaction of these molecules. Examples of anti-CTLA-4 antibodies are described in U.S. Pat. Nos. 5,811,097; 5,811,097; 5,855,887; 6,051,227; 6,207,157; 6,682,736; 6,984,720; and 7,605,238. One anti-CDLA-4 antibody is tremelimumab, (ticilimumab, CP-675,206). In one embodiment, the anti-CTLA-4 antibody is ipilimumab (also known as 10D1, MDX-D010) a fully human monoclonal IgG antibody that binds to CTLA-4. Ipilimumab is marketed under the name Yervoy™ and has been approved for the treatment of unresectable or metastatic melanoma.

Another immune checkpoint protein is programmed cell death 1 (PD-1). PD-1 limits the activity of T cells in peripheral tissues at the time of an inflammatory response to infection and to limit autoimmunity PD-1 blockade in vitro enhances T-cell proliferation and cytokine production in response to a challenge by specific antigen targets or by allogeneic cells in mixed lymphocyte reactions. A strong correlation between PD-1 expression and response was shown with blockade of PD-1 (Pardoll, Nature Reviews Cancer, 12: 252-264, 2012). PD-1 blockade can be accomplished by a variety of mechanisms including antibodies that bind PD-1 or its ligand, PD-L1. Examples of PD-1 and PD-L1 blockers are described in U.S. Pat. Nos. 7,488,802; 7,943,743; 8,008,449; 8,168,757; 8,217,149, and PCT Published Patent Application Nos: WO03042402, WO2008156712, WO2010089411, WO2010036959, WO2011066342, WO2011159877, WO2011082400, and WO2011161699. In certain embodiments the PD-1 blockers include anti-PD-L1 antibodies. In certain other embodiments the PD-1 blockers include anti-PD-1 antibodies and similar binding proteins such as nivolumab (MDX 1106, BMS 936558, ONO 4538), a fully human IgG4 antibody that binds to and blocks the activation of PD-1 by its ligands PD-L1 and PD-L2; pembrolizumab (MK-3475 or SCH 900475), a humanized monoclonal IgG4 antibody against PD-1; CT-011 a humanized antibody that binds PD-1; AMP-224 is a fusion protein of B7-DC; an antibody Fc portion; BMS-936559 (MDX-1105-01) for PD-L1 (B7-H1) blockade; and cemiplimab-rwlc (anti-PD-1 antibody).

In a particular embodiment, the anti-PD-1 antibody (or antigen binding antibody fragment thereof) comprises 1, 2, 3, 4, 5, or all 6 the CDR amino acid sequences of SEQ ID NOs: 1-6 (representing HC CDR1, HC CDR2, HC CDR3, LC CDR1, LC CDR2, and LC CDR3, in that order). In specific embodiments, the anti-PD-1 antibody (or antigen binding antibody fragment thereof) comprises all 6 of the CDR amino acid sequences of SEQ ID NOs: 1-6. In other embodiments, the anti-PD-1 antibody (or antigen binding antibody fragment thereof) comprises (a) the heavy chain variable region amino acid sequence in SEQ ID NO: 7, or a variant sequence thereof which differs by only one or two amino acids or which has at least or about 70% sequence identity, or (b) the light chain variable region amino acid sequence in SEQ ID NO: 8 or a variant sequence thereof which differs by only one or two amino acids or which has at least or about 70% sequence identity. In an exemplary embodiment, the anti-PD-1 antibody (or antigen binding antibody fragment thereof) comprises the heavy chain variable region amino acid sequence in SEQ ID NO: 7 and the light chain variable region amino acid sequence in SEQ ID NO: 8. In other embodiments, the anti-PD-1 antibody (or antigen binding antibody fragment thereof) comprises (a) the heavy chain amino acid sequence of SEQ ID NO: 9 or a variant sequence thereof which differs by only one or two amino acids or which has at least or about 70% sequence identity; or (b) the light chain amino acid sequence of SEQ ID NO: 10 or a variant sequence thereof which differs by only one or two amino acids or which has at least or about 70% sequence identity. In an exemplary embodiment, the anti-PD-1 antibody (or antigen binding antibody fragment thereof) comprises the heavy chain amino acid sequence of SEQ ID NO: 9 and the light chain amino acid sequence of SEQ ID NO: 10.

In a particular embodiment, the anti-PD-1 antibody is encoded by one or more nucleic acid sequences (or an antigen binding portion thereof). In exemplary aspects, the antibody comprises 1, 2, 3, 4, 5, or all 6 CDRs encoded by the nucleic acid(s) of SEQ ID NOs: 11-16 (representing HC CDR1, HC CDR2, HC CDR3, LC CDR1, LC CDR2, and LC CDR3, in that order). In another exemplary aspect, the antibody comprises all 6 CDRs encoded by the nucleic acids of SEQ ID NOs: 11-16. In some embodiments, the anti-PD-1 antibody (or an antigen binding portion thereof) comprises (a) a heavy chain variable region encoded by SEQ ID NO: 17 or a variant sequence thereof which differs by only 1, 2, 3, 4, 5, or 6 nucleic acids or which has at least or about 70%, 85%, 90%, or 95% sequence identity, or (b) a light chain variable region encoded by SEQ ID NO: 18 or a variant sequence thereof which differs by only 1, 2, 3, 4, 5, or 6 nucleic acids or which has at least or about 70%, 85%, 90%, or 95% sequence identity. In an exemplary embodiment, the anti-PD-1 antibody (or an antigen binding portion thereof) comprises a heavy chain variable region encoded by SEQ ID NO: 17 and a light chain variable region encoded by SEQ ID NO: 18. In other embodiments, the anti-PD-1 antibody (or an antigen binding portion thereof) comprises (a) a heavy chain encoded by SEQ ID NO: 19 or a variant sequence thereof which differs by only 1, 2, 3, 4, 5, or 6 nucleic acids or which has at least or about 70%, 85%, 90%, or 95% sequence identity, or (b) a light chain encoded by SEQ ID NO: 20 or a variant sequence thereof which differs by only 1, 2, 3, 4, 5, or 6 nucleic acids or which has at least or about 70%, 85%, 90%, or 95% sequence identity. In an exemplary embodiment, the anti-PD-1 antibody (or an antigen binding portion thereof) comprises a heavy chain encoded by SEQ ID NO: 19 and a light chain encoded by SEQ ID NO: 20.

Other immune-checkpoint inhibitors include lymphocyte activation gene-3 (LAG-3) inhibitors, such as IMP321, a soluble Ig fusion protein (Brignone et al., 2007, J. Immunol. 179:4202-4211). Other immune-checkpoint inhibitors include B7 inhibitors, such as B7-H3 and B7-H4 inhibitors. In particular, the anti-B7-H3 antibody MGA271 (Loo et al., 2012, Clin. Cancer Res. July 15 (18) 3834). Also included are TIM3 (T-cell immunoglobulin domain and mucin domain 3) inhibitors (Fourcade et al., 2010, J. Exp. Med. 207:2175-86 and Sakuishi et al., 2010, J. Exp. Med. 207:2187-94).

### Kits

Kits for use by medical practitioners comprising an oncolytic virus of the present invention (e.g., a herpes simplex 1 virus, wherein the herpes simplex virus lacks functional ICP34.5 genes, lacks a functional ICP47 gene and comprises a gene encoding human GM-CSF—such as talimogene laherparepvec) and a package insert or label with directions to treat melanoma, breast cancer (e.g., triple negative breast cancer), renal cancer, bladder cancer, colorectal cancer, lung cancer, naso-pharyngeal cancer, pancreatic cancer, liver cancer, non-melanoma skin cancers, neuroendocrine tumors, T cell lymphoma (e.g., peripheral), or cancers of unknown primary origin, pediatric solid tumors with unresectable skin lesions using the oncolytic virus as a neoadjuvant therapy. In some embodiments, the cancer is a Stage 3a, 3b, 3c, 3d, or 41a cancer. In a particular embodiment, the cancer is melanoma (e.g., a Stage 2 melanoma). In a particular embodiment, the cancer is melanoma (e.g., a Stage 3a, 3b, 3c, 3d, or 41a melanoma). In a particular embodiment, the oncolytic virus is talimogene laherparepvec, RP1, RP2, or RP3. In another embodiment, the oncolytic virus is talimogene laherparepvec.

In other embodiments, the present invention relates to kits comprising: [1] a herpes simplex virus lacking functional ICP34.5 genes, lacking a functional ICP47 gene, and comprising a gene encoding human GM-CSF; and [2] a package insert or label with directions to treat a cancer by administering a combination of an oncolytic virus and a first checkpoint inhibitor; surgically removing any remaining tumor; and administering a second checkpoint inhibitor, wherein said first and second checkpoint inhibitors may be the same or different. In some embodiments, the oncolytic virus is talimogene laherparepvec, RP1, RP2, or RP3. In another embodiment, the oncolytic virus is talimogene laherparepvec. In some embodiments, the first and second checkpoint inhibitor may be independently selected from the list comprising a CTLA-4 blocker, a PD-1 blocker, and a PD-L1 blocker. In some embodiments, the CTLA-4 blocker is an anti-CTLA-4 antibody, the PD-1 blocker is an anti-PD-1 antibody, and the PD-L1 blocker is an anti-PD-L1 antibody. The CTLA-4 blocker may be ipilimumab. The PD-1 blocker may be nivolumab, pembrolizumab, CT-011, AMP-224, cemiplimab, or an anti-PD-1 antibody comprising any one or more of SEQ ID NOs: 1-10. The PD-L1 blocker may be atezolizumab, avelumab, durvalumab, or BMS-936559.

In other embodiments, the present invention relates to methods of manufacturing such kits.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. All patents and other publications identified are expressly incorporated herein by reference in their entirety.

### EXAMPLES

The following examples are provided to illustrate specific embodiments or features of the present invention and are not intended to limit its scope.

### Example 1: A Phase 2, Multicenter, Randomized, Open-Label Trial Assessing the Efficacy and Safety of Talimogene Laherparepvec Neoadjuvant Treatment Plus Surgery Versus Surgery Alone for Resectable, Stage 3B to 4M1a Melanoma

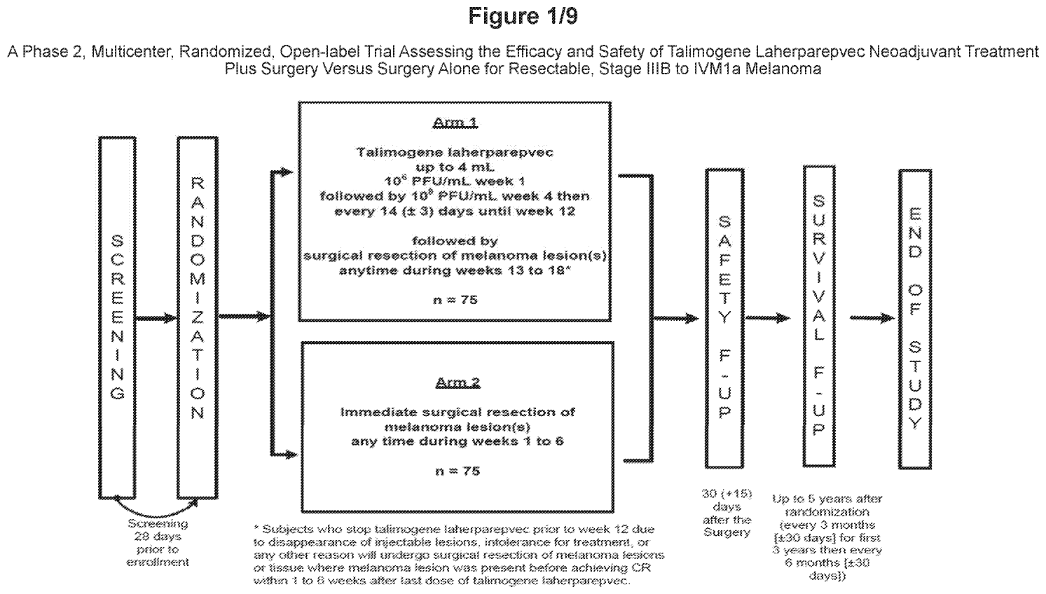

Patients with resectable Stage 3B/C/4M1a MEL, ≥1 injectable cutaneous, subcutaneous, or nodal lesions ≥10 mm, and no systemic treatment 3 months prior were randomized 1:1 to 6 doses/12 weeks of talimogene laherparepvec followed by surgery during weeks 13-18 (Arm 1) vs upfront surgery during weeks 1-6 of the study (Arm 2). See schema in FIG. 1. Talimogene laherparepvec was given at standard dosing until surgery, no injectable tumors, or intolerance. An analysis was conducted on the ITT set to estimate a between-group difference in 1-yr RFS. An RFS event was defined as the first of local, regional or distant recurrence of melanoma or death due to any case, after surgery. Patients not confirmed to be disease-free post-surgery (i.e., did not have R0 surgical outcome) or withdrew prior to surgery were considered an event at randomization for RFS. In a sensitivity analysis, RFS was calculated from randomization to the date of the event removing the without consideration of R0 surgical outcome.

150 patients were randomized (76 Arm 1, 74 Arm 2). 75% in Arm 1 and 93% in Arm 2 had surgery as planned. R0 rates were 42.1% (Arm 1) vs 37.8% (Arm 2). R1 rates (Arm 1 vs Arm 2, respectively) were 51.4% vs 31.6%; R2 rates were 4.1% vs 1.3%. At 1 year, 33.5% of patients in Arm 1 and 21.9% of patients in Arm 2 remained recurrence free (HR 0.73, P=0.048). OS rates at 1 year were 95.9% patients in Arm 1 and 85.8% patients in Arm 2 (HR 0.47, P=0.078). From the sensitivity analysis, 55.8% of patients in Arm 1 and 39.3% in Arm 2 remain recurrence free at the 1 year mark (HR 00.63, P=0.0024).

At 1 year, neoadjuvant talimogene laherparepvec demonstrated improved recurrence-free survival vs surgery alone, 55.8% vs 39.3%%, respectively, HR 00.63, P=0.0024. 95.9% pts in Arm 1 and 85.8% pts in Arm 2 were alive after 1 yr (HR 0.47, P=0.078). 2-year overall survival rates were 88.9% in Arm 1 and 77.4% in Arm 2 (HR: 0.49, P=0.050)

These results indicate that [1] neoadjuvant talimogene laherparepvec improves 2-year RFS and OS in resectable stage IIIB-IVM1a melanoma; and [2] neoadjuvant oncolytic virus therapy (e.g., talimogene laherparepvec) can be used to, e.g., reduce the amount and/or length of adjuvant therapy.

In addition, in Arm 1, talimogene laherparepvec treatment resulted in a 3-fold increase (P<0.001) in intratumoral CD8⁺ cells and an increase in PD-L1 (P≤0.05). Both the mean CD8⁺ density and PD-L1 H-Score in Arm 1 after treatment were significantly higher than those in Arm 2 (P<0.001 for both comparisons; See FIG. 8). Increased intratumoral CD8+ density post-treatment correlated with longer RFS and OS (See FIG. 9). These results indicate that T-cell influx and PD-L1 upregulation after talimogene laherparepvec treatment support a role for the adaptive immune system.

### Objectives

### Primary Objectives:

To estimate the treatment effect of neoadjuvant talimogene laherparepvec plus surgery compared to surgery alone on recurrence-free survival (RFS).

### Secondary Objectives:

To estimate the effect of neoadjuvant talimogene laherparepvec plus surgery compared to surgery alone on 1-year, 2-year, 3-year, and 5-year RFS
To estimate the effect of neoadjuvant talimogene laherparepvec plus surgery compared to surgery alone on rate of histopathological tumor-free margin (R0) surgical resection
To estimate the effect of neoadjuvant talimogene laherparepvec on rate of pathological complete response (pCR)
To estimate the effect of neoadjuvant talimogene laherparepvec plus surgery compared to surgery alone on local recurrence-free survival (LRFS), regional recurrence-free survival (RRFS), and distant metastases-free survival (DMFS)
To estimate the effect of neoadjuvant talimogene laherparepvec plus surgery compared to surgery alone on 1-year, 2-year, 3-year, 5-year, and overall survival (OS)
To estimate response to neoadjuvant talimogene laherparepvec overall and separately in injected and uninjected lesions during treatment (Arm 1 only)
To evaluate the safety of neoadjuvant talimogene laherparepvec plus surgery compared to surgery alone

### Results:

TABLE 2
Patient Treatment Status (from Interim Analysis 1)

| | Arm 1: Talimogene | | | |
|---|---|---|---|---|
| | Laherparepvec + | | Arm 2: Surgery | |
| | Surgery | | alone | |
| | (n = 76) | | (n = 74) | |
| Mean (SD) number of treatment visits where | 5.4 | (1.2) | N/A | |
| patients received talimogene laherparepvec | | | | |
| doses | | | | |
| Patients who never received talimogene | 3 | (3.9) | NA | |
| laherparepvec - n (%) | | | | |
| Patients who discontinued talimogene | 16 | (21.1) | N/A | |
| laherparepvec - n (%) | | | | |
| Disease progression | 7 | (9.2) | N/A | |
| No injectable lesions | 4 | (5.3) | N/A | |
| Patient request | 2 | (2.6) | N/A | |
| Adverse event | 1 | (1.3) | N/A | |
| Ineligibility determined | 1 | (1.3) | N/A | |
| Requirement for alternative therapy | 1 | (1.3) | N/A | |
| Patients who did not receive protocol defined | 19 | (25.0) | 5 | (6.8) |
| surgery - n (%) | | | | |
| Disease progression | 11 | (14.5) | 0 | (0.0) |
| Patient request | 4 | (5.3) | 4 | (5.4) |
| Decision by sponsor | 2 | (2.6)^{a} | 0 | (0.0) |
| Ineligibility determined | 1 | (1.3) | 1 | (1.4) |
| Requirement for alternative therapy | 1 | (1.3) | 0 | (0.0) |

TABLE 3
Interim Analysis #1 Efficacy Results Intent to Treat Analysis (All Subjects)

| | | Talimogene | |
|---|---|---|---|
| | Surgery | Laherparepvec + | |
| | Only Arm | Surgery Arm | |
| | (N = 74) | (N = 76) | Treatment Arm |
| | n (%) | n (%) | Difference |
| Response to | NA | Response Rate | NA |
| Neoadjuvant | | (CR/PR): 10 (11.2) | |
| Treatment | | 80% CI: (8.3, 19.5) | |
| | | Disease Control Rate | |
| | | (CR/PR/SD): 31 (40.8) | |
| | | 80% CI: (33.2, 48.8) | |
| R0 Resection | 28 (37.8) | 32 (42.1) | Difference: 4.3% |
| Rate | 80% CI: (30.3, 45.9) | 80% CI: (34.4, 50.1) | 80% CI: (−6.9, 15.3) |
| | | | p-value: 0.594 |
| 1 R1 Resection | 38 (51.4) | 24 (31.6) | Difference: −19.8% |
| Rate | | | |
| R2 Resection | 3 (4.1) | 1 (1.3) | Difference: −2.7% |
| Rate | | | |
| pCR | 2 (2.7) | 13 (17.1) | Difference: 11 (14.4)% |
| | 80% CI: (0.7, 7.0) | 80% CI: (11.6, 24.0) | 80% CI: (7.4, 21.6) |
| | | | p-value: 0.003* |

*Confidence intervals for differences in rates and for p-values calculated using the Clopper-Pearson method

TABLE 4
Efficacy for intent to treat patients

| | | | Treatment | Overall | Overall |
|---|---|---|---|---|---|
| | Arm 1 | Arm 2 | Difference | Unstratified | Unadjusted |
| | (TVEC + Surgery) | (Surgery alone) | KM Estimate | Hazard Ratio | Log Rank |
| | KM Estimate | KM Estimate | (Arm 1 − Arm 2) | (Arm 1/Arm 2) | Test P- |
| | (N = 76) | (N = 74) | (80% Ci) | (80% CI) | value |
| Recurrence | 33.5% | 21.9% | 11.5% (2.0%, 21.1%) | 0.73 (0.56, 0.93) | 0.048 |
| Free Survival | | | | | |
| (RFS) at 1 year | | | | | |
| Local | 42.0% | 31.1% | 10.9% (0.7%, 21.0%) | 0.81 (0.62, 1.05) | 0.218 |
| Recurrence- | | | | | |
| Free Survival | | | | | |
| (LRFS) at 1 | | | | | |
| year | | | | | |
| Regional | 43.4% | 30.8% | 12.6% (2.4%, 22.8%) | 0.77 (0.59, 1.00) | 0.120 |
| Recurrence- | | | | | |
| Free Survival | | | | | |
| (RRFS) at 1 | | | | | |
| year | | | | | |
| Distant | 34.9% | 24.7% | 10.2% (0.4%, 20.0%) | 0.74 (0.57, 0.95) | 0.062 |
| Metastases- | | | | | |
| Free Survival | | | | | |
| (DMFS) at 1 | | | | | |
| year | | | | | |
| Recurrence | 55.8% | 39.3% | 16.5% (6.0%, 27.1%) | 0.63 (0.47, 0.83) | 0.024 |
| Free Survival | | | | | |
| (RFS) at 1 year | | | | | |
| (sensitivity) | | | | | |
| Recurrence | 29.5% | 16.5% | 13.1% (4.0%, 22.1%) | 0.75 (0.58, 0.96) | 0.07 |
| Free Survival | | | | | |
| (RFS) at 2 year | | | | | |
| Local | 36.5% | 27.5% | 9% (−1.0%, 19.0%) | 0.83 (0.64, 1.08) | 0.29 |
| Recurrence- | | | | | |
| Free Survival | | | | | |
| (LRFS) at 2 | | | | | |
| year | | | | | |
| Regional | 39.2% | 25.4% | 13.8% (3.8%, 23.8%) | 0.77 (0.59, 1.01) | 0.12 |
| Recurrence- | | | | | |
| Free Survival | | | | | |
| (RRFS) at 2 | | | | | |
| year | | | | | |
| Distant | 33.7% | 19.5% | 14.1% (4.7%, 23.6%) | 0.74 | 0.069 |
| Metastases- | | | | | |
| Free Survival | | | | | |
| (DMFS) at 2 | | | | | |
| year | | | | | |
| Recurrence | 50.5% | 30.2% | 20.3% (9.9%, 30.7%) | 0.66 (0.50, 0.87) | 0.038 |
| Free Survival | | | | | |
| (RFS) at 2 year | | | | | |
| (sensitivity) | | | | | |
| Overall | 88.9% | 77.4% | 11.5% (3.5%, 19.4%) | 0.49 (0.30, 0.79) | 0.050 |
| Survival (2 | | | | | |
| year landmark) | | | | | |

### Example 2: A Phase 3, Multicenter, Placebo Controlled, Randomized, Multi-Center Clinical Trial Designed to Evaluate the Efficacy and Safety of Talimogene Laherparepvec in Combination with a PD-1 Inhibitor in the Neoadjuvant Setting Followed by Anti-PD-1 Therapy in the Adjuvant Setting in Subjects with Resectable Melanoma (Stage IIIB-IVM1a)

Approximately 700 eligible subjects are randomized 1:1 into the following treatment arms:
Arm A: Subjects receive talimogene laherparepvec+PD-1 inhibitor in the neoadjuvant setting prior to resection.
Arm B: Subjects receive placebo+PD-1 inhibitor in the neoadjuvant setting prior to resection.

Subjects in Arm A receive 3 doses of talimogene laherparepvec (Week 1: up to 4 mL at 10⁶ PFU/mL, Week 4, 7: up to 4 mL at 10⁸ PFU/mL) and anti-PD-1 therapy using treatment regimens known in the art. Subjects in Arm B receive placebo and anti-PD-1 therapy at Weeks 1, 4, and 7 in the neoadjuvant setting.

All subjects undergo resection at week 10, followed by anti-PD-1 therapy in the adjuvant setting for 1 year. Subjects undergo radiographic assessment prior to resection, and every 3 months after resection to evaluate the tumor response assessed by an independent reviewer. The primary endpoint is event free survival (EFS) and key secondary endpoints are overall survival (OS), disease free survival (DFS), pathologic complete response (pCR), and tumor response (RECIST 1.1) endpoints (overall response rate (ORR), complete response (CR), partial response (PR), stable disease (SD), disease progression (PD)). The clinical trial follows subjects for 5 years.

In this study, the stage of disease may be expanded to include stage 2 resectable melanoma. In addition, pCR following surgery may be used to guide the adjuvant therapy in one arm of the study.

The duration of adjuvant anti-PD-1 therapy may be adjusted to less than 1 year.

In addition, co-primary endpoints of OS and EFS/DFS may be evaluated.

1. A method for the treatment of cancer comprising:
administering a combination of an oncolytic virus and a first checkpoint inhibitor;
surgically removing any remaining tumor; and
administering a second checkpoint inhibitor,
wherein said first and second checkpoint inhibitors may be the same or different.

2. The method according to claim 1, wherein said oncolytic virus an adenovirus, reovirus, measles, herpes simplex, Newcastle disease virus, senecavirus, or vaccinia virus.

3. The method according to claim 2, wherein said oncolytic virus is an adenovirus, reovirus, herpes simplex, Newcastle disease virus, or vaccinia virus.

4. The method according to claim 2, wherein said oncolytic virus is a herpes simplex virus.

5. The method according to claim 4, wherein said herpes simplex virus is a herpes simplex 1 virus (HSV-1).

6. The method according to claim 5, wherein said HSV1 is modified such that it:
lacks functional ICP34.5 genes;
lacks a functional ICP47 gene; and
comprises a gene encoding a heterologous gene.

7. The method according to claim 6, wherein said heterologous gene is a cytokine.

8. The method according to claim 7, wherein said cytokine is GM-CSF.

9. The method according to claim 1, wherein said oncolytic virus is talimogene laherparepvec, RP1, RP2, or RP3.

10. The method according to claim 1, wherein said first and second checkpoint inhibitor are independently selected from the list comprising: a CTLA-4 blocker, a PD-1 blocker, and a PD-L1 blocker.

11. The method according to claim 10, wherein said CTLA-4 blocker is an anti-CTLA-4 antibody, said PD-1 blocker is an anti-PD-1 antibody, and said PD-L1 blocker is an anti-PD-L1 antibody.

12. The method according to claim 11, wherein said CTLA-4 blocker is ipilimumab.

13. The method according to claim 11, wherein said PD-1 blocker is selected from the list comprising: nivolumab, pembrolizumab, CT-011, AMP-224, and cemiplimab.

14. The method according to claim 11, wherein said PD-L1 blocker is selected from the list comprising: atezolizumab, avelumab, durvalumab, and BMS-936559.

15. The method according to claim 1, wherein said cancer is melanoma, breast cancer (e.g., triple negative breast cancer), renal cancer, bladder cancer, colorectal cancer, lung cancer, naso-pharyngeal cancer, pancreatic cancer, liver cancer, non-melanoma skin cancers, neuroendocrine tumors, T cell lymphoma (e.g., peripheral), or cancers of unknown primary origin, pediatric solid tumors with unresectable skin lesions.

16. The method according to claim 15, wherein said cancer is Stage 2, 3a, 3b, 3c, 3d, or 41a melanoma.

17. A kit comprising:
a herpes simplex virus lacking functional ICP34.5 genes, lacking a functional ICP47 gene, and comprising a gene encoding human GM-CSF; and
a package insert or label with directions to treat a cancer by:
administering a combination of an oncolytic virus and a first checkpoint inhibitor;
surgically removing any remaining tumor; and
administering a second checkpoint inhibitor,
wherein said first and second checkpoint inhibitors may be the same or different.

18. A method of manufacturing the kit of claim 17.

19. A method for the treatment of cancer comprising:
administering an oncolytic virus;
surgically removing any remaining tumor; and
administering a checkpoint inhibitor.

20. The method according to claim 19, wherein said oncolytic virus an adenovirus, reovirus, measles, herpes simplex, Newcastle disease virus, senecavirus, or vaccinia virus.

21. The method according to claim 20, wherein said oncolytic virus is an adenovirus, reovirus, herpes simplex, Newcastle disease virus, or vaccinia virus.

22. The method according to claim 20, wherein said oncolytic virus is a herpes simplex virus.

23. The method according to claim 22, wherein said herpes simplex virus is a herpes simplex 1 virus (HSV-1).

24. The method according to claim 23, wherein said HSV1 is modified such that it:
lacks functional ICP34.5 genes;
lacks a functional ICP47 gene; and
comprises a gene encoding a heterologous gene.

25. The method according to claim 24, wherein said heterologous gene is a cytokine.

26. The method according to claim 25, wherein said cytokine is GM-CSF.

27. The method according to claim 19, wherein said oncolytic virus is talimogene laherparepvec, RP1, RP2, or RP3.

28. The method according to claim 19, wherein said checkpoint inhibitor is selected from the list comprising: a CTLA-4 blocker, a PD-1 blocker, and a PD-L1 blocker.

29. The method according to claim 28, wherein said CTLA-4 blocker is an anti-CTLA-4 antibody, said PD-1 blocker is an anti-PD-1 antibody, and said PD-L1 blocker is an anti-PD-L1 antibody.

30. The method according to claim 29, wherein said CTLA-4 blocker is ipilimumab.

31. The method according to claim 29, wherein said PD-1 blocker is selected from the list comprising: nivolumab, pembrolizumab, CT-011, AMP-224, and cemiplimab.

32. The method according to claim 29, wherein said PD-L1 blocker is selected from the list comprising: atezolizumab, avelumab, durvalumab, and BMS-936559.

33. The method according to claim 19, wherein said cancer is melanoma, breast cancer (e.g., triple negative breast cancer), renal cancer, bladder cancer, colorectal cancer, lung cancer, naso-pharyngeal cancer, pancreatic cancer, liver cancer, non-melanoma skin cancers, neuroendocrine tumors, T cell lymphoma (e.g., peripheral), or cancers of unknown primary origin, pediatric solid tumors with unresectable skin lesions.

34. The method according to claim 33, wherein said cancer is Stage 2, 3a, 3b, 3c, 3d, or 41a melanoma.

35. A kit comprising:
a herpes simplex virus lacking functional ICP34.5 genes, lacking a functional ICP47 gene, and comprising a gene encoding human GM-CSF; and
a package insert or label with directions to treat a cancer by:
administering an oncolytic virus;
surgically removing any remaining tumor; and
administering a checkpoint inhibitor.

36. A method of manufacturing the kit of claim 35.
